SEQUENCE LISTING (15 sequences)

SEQ ID NO: 1 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 2 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 3 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 4 - DNA, 20 bp, Artificial Sequence
  synthetic
gtgaatgaggatgagtgatg

SEQ ID NO: 5 - DNA, 7382 bp, Mus musculus
gtgaaatgaggatggcaacgccatcttctgcgccctctgtgcgcaacacagagaaacgcaaaaacaaaaagrcttcatctaargctagygtctccttyggagcacctagccttctctcttcggagagtgaagatgaagttmaytayatgacccctcctgagcaggaagctcagcccggcrccctcgcggcccttcatgctgatgggccgcacgccgggctccccgtgacgcgaagtgatgcacgcgtgctgatcttcaatgagtgggaggagaggaagaagtccgagccgtggctacggctggacatgtctgacaaggccatcttccgccgctaccctcatctgcgrcctaaggaagacaaggcygatgcgccctccyatgcggaggacgccatggatgcaagggagccygtggtgggrtccatycttgagcaggatgaccayaagttctaccactactctgtctacatcggcaacggtatggtgatgggtgtcaacaaccccggcgccgccgtttgccaggctgtgattgatgtggaraagctccacctttggtggaggccagtytgggaacctcgccaaccyctcgacccggctgagttgaggaagtgtgtyggcatgaccgtcccytacgtggccaccactgtcaattgctaccaggtctgctgctggattgttgggatcaaggacacctggctgaagagrgcgaagatatccagagattcgcccttctacagcccygtccaggactggaacattgatccccaggagcccttcatcccgtccaagctcaggatggtttctgatggcatcytagtggctctctcaacggtgattggtcggccgatcaagaacctgctggcatcmgtgaagccgctcaacattctgaacatcgtgttgagytgtgactggactttctcgggcatagtcaacgccctgatcctccttgctgagctatttgacatcttttggactccccctgatgtcaccaactggatgatctccatctttggggaatggcaagccgaggggcccttcgaccttgccctggacgttgtgcccaccctgcttggtgggattggcatggccttcggcctgacgtctgaraccatcgggcgtaagctcgcttccaccaactcagccctcaaggccgcccaggagatgggcaagtttgcaattgaggtyttcaagcagatcatggcatggatttggccttctgaggacccggtgcctgctctgctttccaacatggagcaggcggtcatcaagaatgagtgccagcttgagaaccagctcacagccatgttgcgggatcgcaacgctggggccgagttcctgaaagcacttgatgaagaagaacaagaggtccgcaggattgcggccaagtgcgggaactccgccaccacgggcaccaccaacgccctactggctaggatyagcatggctcgtgcggccttcgagaaggcccgcgctgagcagacctcccgggttcgrcccgtggtgatcatggtatctggcaggcccgggatcgggaaaacctgtttctgtcaaaacctggcaaagaggattgccgcctcccttggrgatgagacctcagtcggcatcataccacgtgctgacgtggaccactgggatgcctacaarggcgctagggtggtcctytgggatgatttcggcatggacaacgtggtgaaggacgctctgcggctgcagatgcttgctgacacatgccccgtcacgcttaactgtgacagaattgagaacaagggkaagatgtttgattcccaggtcatcatcattaccaccaaccagcagaccccagtgccyctggattatgtcaacctggaggcggtgtgccgccgcatagatttcctggtctatgctgagagtcctgtggtggatgccgctcgggccagatcacctggcgatgtggctgccgttaargccgccatgaggccagattacagccacatcaacttcattctggccccacagggtggmtttgaccggcagggtaataccccctatggsaagggcgtcaccaagatcatcggcgccaccgcgctctgtgcaagagcggttgctctcgtccatgagcgccatgatgactttggccttcagaacaaggtctatgattttgatgctggcaaggtgaccgcctttaaggccatggcggctgatgccggcatyccytggtacaagatggcrgcratyggctryaaggccatgggctgcacctgtgtggaggaggccatgaatttgctgaaggactatgaggtggccccstgccaagtgatctacaayggggccacctacaatgtcagctgyatcaarggggcccccatggtwgagaagrtcaaggagccygagytgcccaagacaytggtcaactgtgtcagragratcaaggaggcscgcctccgytgctactgcaggatggccacagatgtcatcacttcyatcytgcaggcggctggracggcyttctctatytaccatcarattgagaagaaatctaggccttccttttattgggaccacggttacacctaccgagatggcccaggtgcctttgacatctttgaggatgacaacgatggatggtaccactctgagrgcaagaagggtaagaataagaaaggtcgggggcggcctggtgtyttcaagtcccgtgggctcacggatgaggagtacgatgagttcaagaagcgccgcgaatccaagggcggcaagtactccattgatgactacctcgctgaccgcgagcgagaagargagctccaggagcgagatgaggaggaggccattttcggggacggctttggcctgaaagccacgcgccgctcccgtaaggcagagagagccagacttggcctggtctcgggtggtgacatccgcgcccgcaagccgattgactggaatgtagttggtccctcctgggccgacgatgatcgccaggtcgattacggtgagaagatcaactttgaggccccagtctccatctggtcccgtgttgtccaattcggcacggggtggggcttctgggtcagtggccatgtgttcatcachgccaagcacgtggcaccacccaagggcacggaggtctttggtcgtaagcccgaggaattcactgtcacctccagtggggatttcctdaaataccatttcaccagtgccgtcaggcctgacatccctgccatggttctggagaacggctgccaggagggcgttgttgcctcagtcctcgtcaagagggcttccggcgagatgctcgctctggcggtcaggatgggctcacaggctgccatcaagatcggcaacgctgtggtgcatgggcagaccggcatgctcttaactgggtccaatgccaaggcccaagacctcgggactatcccgggtgactgtggttgcccctatgtttacaagaagggaaacacctgggttgtgattggggtgcatgtggcggctactagatcaggcaacaccgtcattgccgccacccatggtgagcccacacttgaggccctagaattccaggggcccccaatgctcccccgcccctctggcacctatgctggcctccccatcgccgactatggcgacgcccctcccttgagcaccaagaccatgttctggcgcacctcgccagagaagctcccccctggagcctgggagccagcctaccttggctccaaggatgagagggtggacggcccttccttacagcaggtcatgagagaccaactcaagccctactcagagccacgtggcctgctccctccycaggaaattctggacgcggtttgtgatgccatcgagaaccgccttgagaacacccttgagccgcagaagccctggacattcaagaaggcctgygagagyctkgacaagaayaccagcagtggrtacccctaycacaarcagaaragcaaggactggacgggraccgccttcatyggcgagctcggtgaccaggcyacycatgccaacaacatgtatgagatgggtaagtccatgcggcccgtctacacagctgccctcaaggatgagctggtcaagccagacaagatctacaagaagataaagaagaggttgctctggggctctgaccttggcaccatgattcgcgccgcccgcgcttttggccccttctgtgatgccctgaaagagacttgtgttcttaatcctgtyagagtgggtatgtcgatgaacgaagatggccccttcatcttcgcgaggcacgccaayttcagrtaccacatggatgcagattacaccagatgggactccacccagcagagggcyatcttgaagcgcgccggtgacatcatggtgcgtctctcccctgagccagagttggctcgggtggtgatggatgacctcctggccccctcgctgctggacgtcggcgactataagatcgtcgtcgaagaggggctcccgtccgggtgcccctgcaccacgcagctgaayagtctggcccattggatcctgaccctttgtgcaatggttgaagtgacccgwgttgaccccgayatygtgatgcargartctgaattctccttctatggtgatgacgaggtggtctcgaccaacctcgaattggatatgaccaaatacaccatggccctgaagcggtacggtcttctcccgacccgtgcggacaaggaggagggccccctggagcgtcgccagacgctgcagggcatctccttcctgcgccgcgcaatagtcggtgaccagtttggctggtatggtcgcctcgaccgtgctagcattgaccgccagcttctttggacwaaaggacccaatcaccaraacccytttgagactctcccaggacatgctcagagaccctcccaattgatggccctgcttggtgaggctgccatgcatggtgaaaagtactayaggactgtggcttcccgggtctccaaggaggccgcccagagtgggatagaaatggtggtcccacgccaccggtctgttctgcgctgggtgcgctttggaacaatggatgctgagaccccgcaggaacgctcagcagtctttgtgaatgaggatgagtgatggcgcagcgccaaaagccaacggctctgaagccagcggccaggatcttgttcctaccgccgttgaacaggccgtccccattcagcccgtggctggcgcggctcttgccgcccccgccgccgggcaaatcaaccaaattgacccctggatcttccaaaattttgtccaatgcccccttggtgagttttccatttcacctcgaaacaccccaggtgaaatactgtttgatttggccctcgggccagggctcaacccctacctcgcccacctctcagccatgtacaccggctgggttgggaacatggaggttcagctggtcctcgccggcaatgcctttactgctggcaaggtggttgttgcccttgtaccaccctattttcccaaagggtcactcaccactgctcagatcacatgcttcccacatgtcatgtgtgatgtgcgcaccctggagcccattcaactscctcttcttgacgtgcgtcgagttctttggcatgctacccaggatcaggaggaatctatgcgcctggtctgcatgctgtacacgccactccgcacaaacagcccgggtgatgagtcttttgtggtctctggccgccttctttctaagccggcggctgatttcaattttgtatacctgaccccccccattgagagaaccatctaccggatggtcgacttgcccgtgttgcagccgcggctgtgcacgcatgctcgttggccagccccgatttatggcctcctggtggacccatccctcccgtccaayccccaatggcagaatggtagagtgcatgttgatggaaccctcctcggtacgacacctgtctctgggtcctgggtttcctgctttgcggctgaagctgcctaygagtttcagtctggcattggtgaggtggcaactttcaccctgattgagcaggatggctctgcctatgtccctggtgacagggcagcaccccttggctaccccgatttctccgggcaactggagattgaggtgcagactgagaccaccaaagcaggtgacaagctgaaggtgaccaccttygagatggtccttggccccaccaccaacgtggatcaagcgccctaccagggcagggtgtacgcyagcctaacggctgygtcctccctcgatctggtggatggcagggttagggcggttccacgctctgtctttggcttccaagatgtggttcctgagtataatgatggcctccttgtcccccttgcccccccaatyggccccttycttcctggtgaggtgcttctgaggttccggacctacatgcgtcaggttgacagctctgacgccgctgcggaagccatcgactgcgcccttccacaggaattcgtctcgtggtttgcgagtaacggattcacggtgcagtcggaggccctgctccttaggtacaggaacaccctaacagggcagctgctgtttgagtgcaagctctacagcgaaggctacatcgccctgtcctatccgggctcaggaccgctcaccttcccgactgatggcttcttcgaggttgtcagttgggtcccccgcctttatcaattggcctctgtgggaagcttggcaacaggccgaacactcaaacaataatggctggtgccctctttggagcaattggaggtggcctgatgggtataattggcaattccatctcaaatgttcaaaaccttcaggcaaataaacaattggctgctcagcaatttggttayaattcttctttgcttgcaacgcaaattcaggcccagaaggatctcactctgatggggcagcaattcaaccagcagctccaagccaactctttcaagcacgacttggaaatgctcggcgcccaggtgcaagcccaggcgcaggcccagragaatgccatcaacatcaaatcggcacaactccaggccgcgggcttttcaaagtctgacgccattcgcctggcctcggggcagcaaccgacgagggccgtcgactggtcggggacgcggtattacaccgccaaccagccggtcacgggcttctcgggtggcttyaccccaagttacactccaggtaggcaaatggcagtccgccctgtggacacatcccctctaccggtctcaggtgggcgcatgccgtcccttcgtggaggttcctggtctccgcgtgactacacgccacagactcaaggcacctacacgaacggtcggttcgygtccttcccraagatcgggagtagcagggcgtaggttggaagagaaacctttctgtgaaaatgatttctgcttactgctcttttcttttggtagtatttagatgcattt

SEQ ID NO: 6 - DNA, 20 bp, Artificial Sequence
  synthetic
gugaaugaugauggcgucga

SEQ ID NO: 7 - DNA, 7710 bp, Norwalk virus
  Norovirus GI isolate NORO_79_05_07_2014 (1)..(7710)
gtgaatgatgatggcgtcgaaagacgtcgttgcaactaatgttgcaagcaacaacaatgctaacaacactagtgctacatctcggttcttatcgagatttaagggcttaggaggcggcgcaagcccccctagccctataaaaattaaaagtacagaaatggctctggggttaattggcagaacgaccccagaatcaacggggaccgctggcccaccgcccaaacaacagagagaccgacctcctagaactcaggaggaggtccagtacggtatggggtggtctgacaggcccattgaccagaacgtcaaatcatgggaagagcttgacaccacagttaaggaagagatcctagacaaccacaaagaatggtttgacgctggtggtttgggtccttgcacaatgcctccaacatatgaacgggtcagggatgacagtccgcctggtgaacaggttaaatggtccgcacgtgatggagtcaacattggagtggaacgcctcacaacagtgagtgggcctgagtggaatctttgccccttaccccccattgatttgaggaacatggaaccagctagtgaacccactattggagatatgatagaattctacgaaggccacatctatcattactccatatacattgggcaaggtaagacagtcggcgtccattctccacaggcggcattttcagtggctagagtgaccatccagcccatagccgcttggtggagagtttgttacataccccaacccaagcatagactgagttacgaccaactcaaggaactagagaatgagccatggccatacgcggccataactaataattgttttgaattctgctgtcaagtcatgaaccttgaggacacgtggttgcaaaggcgactggtcacgtcgggcagattccaccaccccacccagtcgtggtcacagcagacccctgagttccaacaagatagcaagttagagttggttagggacgccatattggctgcagtgaatggtcttgtttcgcagccctttaagaacttcttgggtaaactcaaacccctcaatgtgcttaacatcctgtctaactgtgattggaccttcatgggggtggtggaaatggtcatactattacttgaactctttggtgtgttctggaacccgcctgatgtatccaattttatagcgtcccttcttcctgatttccatcttcagggacctgaagacttggcacgagatctagtcccagtgattcttggtggtataggattggccattgggttcaccagagacaaagttacaaagatcatgaagagtgctgtggatggtcttcgagctgctacacaactgggacagtatggattagaaatattctcactgctcaagaagtacttctttgggggggaccagactgagcgcaccctcaaaggcattgaggcagcagtcatagatatggaggtactgtcctccacttcagtgacacagctagtgagggacaaacaggcagcaaaggcctatatgaacatcttggacaatgaagaagagaaggccaggaagctctctgctaaaaacgctgacccacatgtgatatcctcaacaaatgccctaatatcgcgcatatccatggcacgatctgcattggccaaggcccaggctgagatgaccagtcgaatgcgaccagttgtcattatgatgtgtggtccacctgggattgggaagaccaaggctgctgagcacctagctaagcgtctagccaatgagatcagaccaggtggtaaggtggggttggttccccgtgaagctgtcgaccactgggacggctatcatggtgaggaagtgatgctgtgggatgactatggcatgacaaaaatacaagacgactgtaataaactccaggccattgctgattcggcccccctcacattaaattgtgataggattgaaaataaaggaatgcagttcgtttcagatgcaatagtcatcaccaccaacgccccaggccccgcccctgtggactttgtcaaccttggaccagtgtgtagacgggtcgactttttggtgtactgctctgccccagaggtggagcagatacggagagtcagccctggcgacacatcagcactgaaagactgcttcaagccagatttctcacatttaaaaatggagctggctccacaaggtgggttcgataatcaagggaacacaccgtttggcaggggcaccatgaagccaacaaccattaatagactcctcatacaagccgtggcccttaccatggaaaggcaggatgagttccagttgcagggaaagatgtatgactttgatgatgacagggtgtcagcgttcaccaccatggcacgtgacaatggcctgggcatcttgagcatggcgggtctaggtaagaagctacgcggtgtcacaacgatggagggcttgaagaatgccctgaagggatacaaaattagtgcgtgcacaataaaatggcaggctaaagtgtactcactagagtcagatggcaacagtgtcaacattaaagaggagaggaacatcttaactcaacaacaacagtcagtgtgtgctgcctctgttgcgctcactcgcctccgggctgcgcgtgcggtggcatacgcgtcatgcatccaatcggctataacctctatactacaaattgctggctcggccctagtggtcaacagagcagtgaagagaatgtttggcacgcgtactgccaccctgtcccttgagggcccccccagagaacacaagtgcagggtccacatggccaaggccgcaggaaaggggcctattggccatgatgatgtggtagaaaagtatgggctttgcgaaactgaggaggacgaagaagtggcccacactgaaatcccttctgccaccatggagggcaagaataaagggaagaacaagaaaggacgtggtcggaagaacaactacaacgccttctcccgcaggggactcaatgatgaagagtacgaagagtacaagaagatacgcgaggagaaaggtggcaattatagcatacaggagtacctagaggataggcaaaggtatgaagaagagctagcagaggttcaagcaggtggagatggaggaatcggggaaactgaaatggaaatccgccacagagtgttctacaaatctaagagtagaaagcatcaccaggaagagcgacgccagctagggctggtaacaggttccgacattcggaagagaaaaccaatcgactggaccccacccaagtcagcatgggcagatgatgagcgtgaggtggattacaatgagaagatcagttttgaggcgccccccactttatggagcagagtgacaaagtttgggtctggatggggtttctgggtcagctctacagtcttcataaccacaacgcacgtcataccaaccagtgcgaaggaattctttggtgaacccctaaccagcatagccatccacagggctggtgagttcactctattcaggttctcaaagaaaattaggcctgacctcacaggtatgatccttgaggagggttgccccgagggcacagtgtgttcagtactaataaaaagggactctggtgaactactgccattggctgtaagaatgggcgcaatagcatcaatgcgtatacagggccgccttgtccatgggcagtccggcatgttgctcaccggggccaatgctaagggcatggaccttggaaccatcccaggagactgtggggctccttatgtctataagagagccaacgactgggtggtctgtggtgtacacgctgctgccaccaaatcaggcaacaccgttgtgtgcgccgttcaggccagtgaaggagaaaccacgcttgaaggcggtgacaaaggtcattatgctggacatgaaataattaagcatggttgtggaccagccctgtcaaccaaaaccaaattctggaaatcatcccccgaaccactaccccctggggtctatgaacccgcctacctcgggggccgggaccctagggtaactggcggtccctcactccaacaggtgttgcgggaccagttaaagccatttgctgagccacgaggacgcatgccagagccaggtctcttggaggccgcagttgagactgtgacttcatcattagagcaggttatggacactcccgttccttggagctatagtgatgcgtgccagtcccttgataagaccactagttctggttttccctaccacagaaggaagaatgacgactggaatggcaccacctttatcagggagttaggggagcaggcagcacacgctaataacatgtatgaacaggctaaaagtatgaaacccatgtacacggcagcacttaaagatgaactagtcaaaccagagaaggtataccaaaaagtgaagaagcgcttgttatggggggcagacttgggcacggtggttcgggccgcgcgggcttttggtccattctgtgatgctataaaatcccacacaatcaaattgcccattaaagttggaatgaattcaattgaggatgggccactgatctatgcagaacattcaaagtataagtaccattttgatgcagattacacagcttgggattcaactcaaaatagacaaatcatgacagagtcattctcaatcatgtgtcggctaactgcatcacctgaactagcttcagtggtggctcaagatttgcttgcaccctcagagatggatgttggcgactatgtcataagagtgaaggaaggcctcccatctggttttccatgtacatcacaggttaatagtataaaccattggttaataactctgtgtgccctttctgaagtaactggtctgtcgccagatgtcatccagtccatgtcatatttctctttctatggtgatgatgaaatagtgtcaactgacatagaatttgatccagcaaaactgacacaagtcctcagagagtatggacttaaacccacccgccccgacaaaagcgagggcccaataattgtgaggaagagtgtggatggtttagtctttttgcgtcgcactatctcccgcgacgccgcaggattccaggggcgactggaccgggcatccattgaaaggcaaatctactggactagaggacccaaccactcagacccttttgagaccctggtgccacatcaacaaaggaaggtccaactaatatcattattgggtgaggcctcactgcatggtgaaaagttttacaggaagatttcaagtaaagtcatccaggagattaaaacagggggccttgaaatgtatgtgccaggatggcaagccatgttccgttggatgcggttccatgaccttggtttgtggacaggagatcgcaatctcctgcccgaatttgtaaatgatgatggcgtctaaggacgcccctcaaagcgctgatggcgcaagcggcgcaggtcaactggtgccggaggttaatacagctgaccccttacccatggaacctgtggctgggccaacaacagccgtagccactgctgggcaagttaatatgattgatccctggattgttaataattttgtccagtcacctcaaggtgagttcacaatctctcctaacaatacccccggtgatattttgtttgatttacaattaggtccacatctaaaccctttcttgtcacatttgtcccaaatgtataatggctgggttgggaacatgagagtcagaattctccttgctgggaatgcattctcagctggaaagattatagtttgttgtgtcccccctggctttacatcttcttctctcaccatagctcaggccacattgtttccccatgtaattgctgatgtgagaacccttgagccaatagaaatgcccctcgaggatgtacgcaatgtcctctatcacaccaatgataatcaaccaacaatgcggttggtgtgtatgctatacacgccgctccgcactggtggggggtctggtaattctgattcctttgtagttgctggcagggttctcacagcccctagtagcgactttagtttcttgttccttgtcccgcctaccatagagcagaagactcgggctttcactgtgcctaatatccccttgcaaaccttgtccaattctaggtttccttccctcatccaggggatgattctgtcccccgatgcatctcaagtggtccaattccaaaatgggcgctgccttatagatggtcaactcctaggcactacacccgctacatcaggacagctgttcagagtaagaggaaagataaatcagggagcccgcacacttaacctcacagaggtggatggtaaaccattcatggcatttgattcccctgcacctgtggggttccccgattttggaaaatgtgattggcatatgagaatcagcaaaaccccaaacaacacaagttcaggtgaccccatgcgcagtgtcagcgtgcaaaccaatgtgcagggttttgtgccacacctgggaagtatacaatttgatgaagtgtttaaccatcccacaggtgactacattggcaccattgaatggatttcccagccatctacaccccctggaacagatattgatctgtgggagatccccgattatggatcatccctttcccaagcagctaatctggcccccccagtattcccccctggatttggtgaggcccttgtgtactttgtttctgctttcccgggccccaataaccgctcagccccgaatgatgtaccctgtcttctccctcaagagtacataacccactttgtcagtgaacaagccccaacgatgggtgacgcagccttactgcattatgtcgaccctgataccaacaggaaccttggggagttcaagctataccctggaggttacctcacctgtgtaccaaatggggtaggtgccgggcctcaacagcttcctcttaatggtgtttttctctttgtttcttgggtgtctcgtttttatcagcttaagcctgtgggaacagccagtacggcaagaggtaggcttggagtgcgccgtatataatggcccaagccatcataggagcaattgccgcgtcagctgcaggctcagcattgggtgcgggcatccaggctggtgccgaggctgcgcttcagagtcaaagataccaacaagacttagccctgcaaaggaatacttttgaacatgacaaggatatgctttcctaccaggtccaggcaagtaatgcacttttggcaaagaatctcaatacccgctattctatgcttgttgcagggggtctttctagtgctgatgcttctcgggctgttgctggggcccctgtaacacaattgattgattggaacggcactcgggttgccgcccccagatcaagtgcaacaactctgaggtctggtggtttcatggcagtccccatgcctgttcaatccaaatctaaggccctgcaatcctctgggttttctaatcctgcttatgacacgtccacagtttcttctaggacttcttcttgggtgcagtcacagaattccctgcgaagtgtgtcaccctttcataggcaggcccttcaaactgtatgggttactccacctgggtctacttcctcttcttctgtttcctcaacaccttatggtgtttttaatacggataggatgccgctattcgcaaatttgcggcgttaatgttgtaatataatgcagcagtgggcactatattcaatttggtttaattagtgaataatttggccattgattagtgttaa

SEQ ID NO: 8 - DNA, 20 bp, Artificial Sequence
  synthetic
guaaaagaaauuugagacaa

SEQ ID NO: 9 - DNA, 7685 bp, Feline calicivirus
  Feline calicivirus strain GX01-13 (1)..(7685)
atgtctcaaactctgagcttcgtgctaaaaacccacagtgtccgtaaggactttgtgcactccgtcaagttaacacttgctcggaggcgcgatcttcagtatctttataacaagcttgcccgctctatacgagcggaggcttgtccatcttgtgctagttacgacgtttgtcctaactgcacctctagtgacattcccgatgatggttcgtcaacaaactcgattccatcttgggatgacgtcacgaaaacttcaacctattccctcttactctccgaggatacatctgatgagcttagccctgatgatttggttaacattgcttcccacatccgtaaggcaatatcctctcagtcgcatcctgccaacaatgagatgtgcaaagaacagctcacctcgttgctgacagtggctgaggccatgttgccccaacgatcgcggtcaacaatcccactgcatcagaaacaccaggcagctcgattggaatggagagaaaaattcttttctaaacctcttgacttcctccttgagaaacttggcatgtctaaggacattctacaaaccactgctatttggaagattgttttggaaaaggcctgctactgtaaatcttatggtgaacaatggtttaatgctgcaaaggcaaagctccgtgagatcaaggaattcgagggaagtactttaaaacctttaattggtgcgtttattgacggactgcggctcatgaccgtcgataatccaaaccctattggcttcttgccaaaattaattggcttagttaaacctctaaatttggcaatgataattgacaaccatgaaaataccatgtcaggatgggttgtaaccctcacagcaatcatggagctgtacaacattactgagtgtacaattgatgtgattacggcgctgatcactggattctatgacaaattggcaaaagctaccaaattttatagtcaggttaaagctttattcactggatttagatcagaggaagtgtcaaattcattttggtacatggcagctgcagtattgtgctaccttatcactggcttgctaccaaacaatggcaggctttcaaaaatcaaggcctgtttgtctggtgcttcgacgctagtatctggtataattgccacacaaaagcttgctgcaatgtttgccacttggaactccgaaacaatagttaatgaactttcagccaggactgttgcgctttcggagcttaacaaccccaccacgacatccgacactgactcagtagaaagactactagaattggctaagatcttacatgaagaaatcaaagttcacacgttgaatccaattatgcaatcatacaacccaattctcagaaatttgatgtcaacattggatggtgtcatcacatcatgcaacaaacgaaaagccattgctaagaagagacctgttccagtatgttatatactaactggtccaccaggttgtgggaaaacaacagctgctttagcattggcaaagaagttgtcagaacaagagccatctgttataaatttggatgtagatcaccatgacacatacactggcaacgaagtctgcatcattgatgaatttgattcgtctgacaaggtcgattatgcaaattttgttattgggatggttaattcggcacccatggtcttaaattgtgacatgcttgaaaacaaggggaagctctttacctctaaatatattataatgacctctaattctgaaactcctgttaagcccggttcaaagcgtgccggtgcattctatcgaagggtcacaatcattgatgtcacaaaccctttggtagagtcacacaagcgcgccagacctggcacctctgttcctcgcagttgctataagaaaaacttctctcatctgtcgcttgctaagcgtggggctgagtgttggagcaaggagtatgtccttgaccccaagggactccagcatcaaagcattaaggcccctccgcccaccttccttaatattgattctcttgctcaaacaatgatacaagatttcacactaaagaacatggcatttgaggcagaggaaggatgcagtgatcaccggtatgggtttatctgccagaaggaggaagtggaaacagttcgcagacttcttaatgcaattagggttaggctcaatgcaactttcacagtctgtgtagggcctgaagcatctagttcagtgggatgtaccgctcacgtcttaacaccagatgagccgttcaatggtaaaagatttgtggtttctcgctgtaatgaggcgtcactatctgcattagaaggcaactgtgtccaaaccgcattgggtgtgtgcatgtccaacaaggatctaacccatttgtgtcatttcataagggggaagattgtcaatgatagtgtcagactggatgaactacccgctaatcaacatgtggtaaccgttaactcggtgtttgatttagcctgggctcttcgccgtcacctgtcactatctggacagttccaagccatcagagccgcatatgatgtgcttactgtccccgataaaatccctgcaatgttaagacactggatggatgagacttcattctctgatgaacatgtcgtaacccaattcgtaacccctggtggtatagtgattcttgaatcatgtgttggtgctcgcatctgggccattggtcacaatgtgatcagggctggaggtatcaccgccacaccgactgggggttgcgtgagattaatgggattgtcggctcatactatgccatggagtgaaatctttagggaactcttctctcttctggggaaaatctggtctagtgttaaagtctccactctagttctcaccgctcttggaatgtacgcatcaagattcagaccaaaatcagaggcaaaaggcaagacaaagagcaaaattggcccctacagaggtcgtggcgttgcccttaccgacgacgagtatgatgaatggagggaacacaatgccactagaaaattggacttatctgttgaagattttctaatgctaaggcatcgcgcagcacttggtgctgatgatgctgatgctgtcaaattcaggtcttggtggagctctagatcaagacttgctgatgatatagaagatgtcaccgtaattggcaagggtggcgttaaacatgagaaaattagaacaaacactctaagagccgttgatcgtggctacgatgtcagctttgctgaagaatctggccctggaaccaaatttcacaagaatgcaattggctctgtcactgatgcttgtggtgaacacaagggatactgtatccatatgggtcatggtgtttacgcttctgttgcccatgtggtgaaaggtgattcattctttcttggtgagaggatctttgacttgaaaactaatggtgaattctgttgctttagaagcacaagggtactcccaagtgcagctcctttcttttctggaaaacccacacgtgacccatggggctctcctgttgctacagagtggaagccaaagccctacacaacaacatctgggaaaattgtagggtgcttcgcaactacatcaactgaaacccaccctggtgattgtggcctgccgtacatcgatgattgtggaagagttacagggctacatacaggatctggaggcccaaagacccctagtgcaaaattaattgttccatatgtccacattgatatgaaggccaaatctgtcactccccaaaagtatgatgttacaaaacctgacatcagctataaaggtttaatttgcaaacaattggacgaaatcagaattataccaaagggaacccggcttcacgtatctcctgctcacgttgatgactacgaagaatgctctcaccaaccagcatccctcggtagtggtgatccccgatgtccaaaatctctgacagctattgttgttgattccttaaaaccttactgtgataaagtggaaggccctcctcatgatatattgcacagagtccagaaaatgctgattgatcacctgtctggattcgtccccatgaacatatcctctgaaacttctatgctatccgcatttcacaaattgaatcatgacacatcttgtggaccttacttaggtggaaggaagaaagatcatatggtaaatggtgaacctgacaaagctctcttggatctcctatcctcaaaatggaaattggcaacacaagggatttccctcccacacgagtacacaattggtttgaaagacgagctgagaccagtggagaaagtcgctgagggaaagaggaggatgatctgggggtgtgatgtcggtgttgctactgtgtgtgctgctgctttcaaagctgttagtgatgcaatcacagcaaatcatcaatatgggcctattcaagttggtatcaatatggatagtcccagtgttgaggcgctgtaccaacggatcaagagctttgccaaagtctttgcagttgattactccaaatgggattcgactcaatcgccccgtgtaagtgctgcctcaattgacatcctgcgatacttctctgacagatcaccaattgttgattcggccacaaatacacttaaaagcccaccagttgctatttttaatggagttgctgttaaggtcacatctggtttgccctccgaaatgcccctcacctctgtgattaactctcttaaccactgtttgtatgttgggtgtgctatcgttcaatctttagaggctaggaatgtccctgtcacatggaatttgttctcctcttttgacatgatgacttatggtgatgatggtgtgtatatgtttccaatgatgtttgctagtgttagtgaccaaatctttggtaacctttctgcttacggcctaaaaccaacccgagttgacaagaccgttggggctattgagccaattgaccctgagtcagttgtctttctaaaaagaacaatctctagaactccccatggtgtccgaggattgttggatcgcagttcaataattaggcagttttactacatcaaaggtgaaaacacagatgattggaaaacccccccaaaaacaatcgatccaacatcccgtggtcagcaactctggaatgcctgcttgtatgctagtcaacatggaagtgagttctacaacaagatttacaaattggctgtgaaggctgttgagtacgaaggactccaccttgaccctccttcttacagttcggctttggaacattacaacagccagttcaatggcgtggaggcgcggtccgatcagatcaatatgagtgatggtaccgccctacactgtgatgtgttcgaagtttgagcatgtgctcaacctgcgctaacgtgctaaaatactatgattgggacccccactttagattggttattaaccccaacaaattcttacccgttggtttctgcaataaccctcttatgtgttgttaccctgaattgcttcctgaatttggaactgtgtgggactgtgatcaatccccacttcaaatctacctagagtcaatccttggtgatgatgagtggtcttcaacctatgaagcaattgaccctgttgtgccaccaatgcactgggacgaagctggtaagatcttccagccacaccctggtgtactaatgcaccacatcattggtgaagtcgcaaaggcatgggatccgaatctgcctcttttccgacttgaggcagacgacagttccgtaacaacgcctgaacagggcaccgctgttggtggtgtgattgctgagcccaatgcacagatggcagcggccgctgatacggctactgggaaaagtgtcgactcagaatgggagaatttcttctcattccacaccagtgtgaattggagcacttctgaaacccaaggaaagattctgtttaaacaatcacttggtcctcttctaaacccttatctggaacatttgtctaagctatatgttgcttggtctgggtctatcgaagttagattttctatctctggttctggtgtctttggggggaagctcgcggctattgtcgtaccgccggggattaatcccgtggcgagcacttcaatgctgcaatacccgcatgtcctatttgatgctcgtcaagtagaacctgtcatttttactattcctgatcttaggaactcgctttaccacttaatgtctgatactgacactacatccttggttattatgatctataatgatttgattaacccttatgctaatgattctaactcctctggatgcattgtcacagtagagactaagcctggacctgacttcaaatttcacctcttgaaaccacctggctcaatgttaacacatggttctgtaccgtcagatttgattccaaaatcatcctcactatggattggcaaccgctattggtctgacatcaccgatttcattgttcgtccatttgtgttccaggcaaatcgtcactttgactttaatcaagagacagctggttggagtactccaagatttcggcccattagtattaccatcagtcaaaaagacggtgcaaaacttggcactgggattgccactgatttcattgtacctggaataccagacggatggccagacacaacaattgcagaagaactcatccccgctggtgactatgccatcacaaattcagccaataatgatattgccacaaaggctgcttacgaggcagcagatgttatcaagaacaacaccaactttagaggtatgtacatttgtggcgctcttcaaagagcttggggagacaagaaaatttccaatactgctttcatcaccaccgctacaatcagtaataactccatcaagccctgtaacaaaattgatcaaacaaagattactgtgttccaaaacaaccatgttggtagtgatgtacaaacatctgatgacacactagccttgcttggttatacggggattggagaagaagccattggggcgaatagggagaaagttgttcgcatcagtgttttgcgtgaggctggtgcacgcggcgggaatcaccctatattttacaaaaactccattaaattaggctatgtaattggatctattgatgtgttcaattctcaaatcttgcacacgtctaggcaattgtctcttaaccattatctgttggctcctgactcttttgctgtttataggattattgactctaatggttcttggtttgacataggtattgattctgatggattctcctttgttggtgtttctaccattcctccgctagagtttccactttctgcctccttcatgggaatacaattggcaaagattcgacttgcctcaaacattaggagtgctatgacaaaattatgaattcaatattaggccttattgactctgtaactaacacagtaagtaaagcacaacaaattgaattagataaagctgcacttggtcaaaatagagaacttgctttaaaacgtattaacttggatcagcaagctcttaataaccaggtgtcgcaatttaacaaacttcttgagcagagggtacagggccctattcagtcagttcgattagctcgtgctgctggattccgggttgacccttactcatacacaaatcaaaatttttatgatgaccaactcaatgcaattagattatcatatagaaatttgtttaaaatgtagaatgaattttataatttggattgattggatgtacctcttcgggctgtcgctgcgcctaaccccaggg

SEQ ID NO: 10 - DNA, 20 bp, Artificial Sequence
  synthetic
gugaucgugauggcuaauug

SEQ ID NO: 11 - DNA, 20 bp, Artificial Sequence
  synthetic
gugaaaauuauggcggcuau

SEQ ID NO: 12 - DNA, 17 bp, Artificial Sequence
  synthetic
gugacuagagcuaugga

SEQ ID NO: 13 - DNA, 20 bp, Artificial Sequence
  synthetic
gugauuuaauuauagagaga

SEQ ID NO: 14 - DNA, 14 bp, Artificial Sequence
  synthetic
aagtattaccagcc

SEQ ID NO: 15 - DNA, 20 bp, Artificial Sequence
  synthetic
gugaaugaggaugagugaug

(19) United States
(12) Patent Application Publication
(10) Pub. No.: US 2022/0002717 A1
(43) Pub. Date: Jan. 6, 2022

(54) PROGRAMMABLE NUCLEASES AND BASE EDITORS FOR MODIFYING NUCLEIC ACID DUPLEXES

(71) Applicant: Regents of the University of Minnesota, Mnneapolis, MN (US)

(72) Inventors: Branden Moriarity, Minneapolis, MN (US); Mitchell Kluesner, Minneapolis, MN (US); Beau Webber, Minneapolis, MN (US)

(21) Appl. No.: 17/290,968

(22) PCT Filed: Nov. 8, 2019
(86) PCT No.: PCT/US19/60492

§ 371 Date: May 3, 2021

Related U.S. Application Data
(60) Provisional application No. 62/757,282, filed on Nov. 8, 2018.

(51) Int. Cl. C12N 15/11; C12N 9/22; C12N 9/24; C12N 9/78; C12N 9/06

(52) U.S. Cl. CPC C12N 15/111; C12N 9/22; C12Y 301/21; C12N 9/2497; C12Y 302/02; C07K 2319/00; C12Y 305/04004; C12N 9/002; C12Y 402/99018; C12N 2310/3513; C12N 2310/20; C12N 9/78

(57) ABSTRACT

Provided herein are methods and compositions for highly precise base editing and single strand nicking. In particular, provided herein are methods for producing a genetically modified cell where the methods employ a universal, highly precise base editor or staggered Cas9 editor for precise base editing with minimal off-target or bystander effects.

### CROSS-REFERENCE TO RELATED APPLICATIONS

This application claims the benefit of U.S. Provisional Application No. 62/757,282, filed Nov. 8, 2018, which is incorporated in its entirety by reference for all purposes.

### REFERENCE TO A SEQUENCE LISTING SUBMITTED VIA EFS-WEB

The content of the ASCII text file of the sequence listing named “920171_00327_ST25.txt” which is 54.1 kb in size was created on Nov. 8, 2019 and electronically submitted via EFS-Web herewith the application is incorporated herein by reference in its entirety.

### BACKGROUND

The world health organization estimates that there are over 10,000 monogenic diseases, affecting millions of people world-wide. Of these monogenic diseases, pathogenic single nucleotide polymorphisms (SNPs) are a major contributor, of which 54% of mutations are due to A:T↔G:C transition mutations. With the advent of CRISPR-Cas9, the correction of mutations that were previously thought to be incurable are now accessible with this powerful and ever-increasingly applied tool. In the replacement of faulty genes, CRISPR-Cas9 has been largely employed to correct mutations via the induction of a double stranded break at the mutated site, followed by repair of the break from a template containing a functional DNA sequence via homology directed repair (HDR). In principle, Cas9 endonuclease is introduced to mutant cells, alongside a programmable guide RNA (gRNA) and a DNA repair template containing the change of interest. The gRNA binds to Cas9 and directs the complex to a mutated site in the genome via the complementarity of the 20 bp protospacer located at the 5′ end of the gRNA. Once bound, the Cas9-gRNA complex induces a double-stranded break at the target DNA. This double stranded break tends to be repaired more frequently via the quasi-stochastic non-homologous end joining (NHEJ) pathway which results in insertion-deletion (indel) mutations. Meanwhile, if a homologous DNA template is present HDR will incorporate the functional, non-pathogenic changes from the template.

Although the use of CRISPR-Cas9 mediated HDR has greatly improved our ability to correct deleterious SNPs with multiple clinical trials on the horizon, this approach is limited by low rates of correction against a backdrop of high rates of deleterious indels. To improve the ratio of HDR over NHEJ repair, a myriad of approaches have been developed, including the use of a dual-nickase strategy to generate 5′ overhangs, which are the preferentially repaired by HDR. As an alternative, over the past two years multiple research groups have fused the programmable specificity of the Cas9-gRNA complex to mutagenic enzymes such as adenosine or cytidine deaminases (termed Base Editors). These base editors produce targeted correction of deleterious SNPs with minimal-to-no double stranded breaks. The Adenosine deaminase Base Editors (ABEs) were engineered via the directed evolution of a heterodimeric TadA bacterial adenosine deaminase to deaminate adenosine in ssDNA, as opposed to TadA's natural substrate of dsRNA.2 Meanwhile, cytidine deaminase Base Editors (BEs) are engineered via the fusion of a natural cytidine deaminase (APOBECs) that acts on ssDNA, as well as the fusion of a Uracil DNA Glycosylase Inhibitor (UGI), which prevents removal of the nascent uracil in the target DNA. In the cell, the base editor complex is brought to the target site by the core Cas9-gRNA complex, where the displaced ssDNA loop (d-loop) wraps around the complex. Adenonsines and cytidines (for ABEs and BEs respectively) within a ˜5 bp window of the d-loop (corresponding to positions 4-9 of the protospacer) are then free to be deaminated by fused deaminase. In the case of ABEs, this yields inosines which behave like guanines and base pair with cytosine in a Watson-Crick fashion, while in the case of BEs, this yields uridines which behave like thymidines in a Watson-Crick fashion. Additional installation of a D10A mutation in Cas9 produces a nickase (“nCas9”) which nicks the non-edited antisense strand, initiating mismatch repair (MMR), whereby the nonedited strand is degraded and repaired using inosine on the edited strand as a template, or using cytidine in the case of BEs. Base editing represents a paradigm shift in gene editing with an unprecedented resolution of single base modification without double-stranded breaks, however there are still limitations of this approach which preclude potential clinical applications. In addition, non-A:T↔G:C transition mutations are not currently amenable to base editing, thus their correction still largely relies on the use of Cas9 mediated HDR, with high deleterious background indels. Thus, if an enzyme could be engineered that produces programmable DSBs consisting of large 5′ overhangs, then these mutations could be more efficiently, and safely corrected by increased HDR repair.

Since the inception of base editing much of the work has focused on approaches to position the target base within a particular position of the editing window either by changing the PAM specificity, engineering the mutagenic domain to have altered processivity or context preference, altering the linker length of the of the mutagenic domain, or changing the mutagenic domain ortholog. While individual changes have accrued modest improvements in controlling which base is edited within the activity window, it has resulted in a large repertoire of modified enzymes which make it difficult to predict which base editor variant is optimal in a particular situation. Furthermore, although these developments have improved the accessibility to correct certain mutations, sub-optimal editing and imprecise editing (where other bases in the window are edited with potentially deleterious effects) remain significant challenges to current base editing methods. Accordingly, there remains a need in the art for a base editing platform that is less modular, more universal, and has the capability of editing the target base with exact precision.

### SUMMARY OF THE DISCLOSURE

In a first aspect, provided herein is a method for producing a genetically modified cell. The method can comprise or consist essentially of: (a) introducing into a cell one or more plasmids, mRNAs, or proteins encoding (i) a universal precise base editor fusion protein comprising a deaminase fused to a Cas9 nuclease domain, wherein the Cas9 nuclease domain comprises a base excision repair inhibitor domain, (ii) synthetic chimeric ssODN-ssORN duplex, wherein at least a portion of the ssORN is complementary to that of the Cas9 d-loop and comprises a nucleotide mismatch recognized by the base editor fusion protein; and (ii) one or more gRNAs having complementarity to a target nucleic acid sequence to be genetically modified; and (b) culturing the introduced cell under conditions that promote modification of the target nucleic acid sequence targeted by the one or more gRNAs, whereby the target nucleic acid sequence is modified by the base editor fusion protein and gRNAs relative to an unmodified cell, and whereby a genetically modified cell is produced. The base editor fusion protein can be an upABE or an upBE. The base editor fusion protein can comprise a dsRNA adenosine deaminase, the nucleotide mismatch is dA:C, and the Cas9 domain is fused to a PCV2 domain. The dsRNA adenosine deaminase can comprise an amino acid substitution of an E to a Q at position 1008, as numbered relative to SEQ ID NO:1. The dsRNA adenosine deaminase can comprise an amino acid substitution of an E to a Q at position 488, as numbered relative to SEQ ID NO:2. The dsRNA adenosine deaminase can comprise the amino acid sequence set forth as SEQ ID NO:3. The base editor fusion protein can be selected from hADAR1d^{E1008Q}-nCas9-PCV2 and hADAR2d^{E88Q}-nCas9-PCV2. The base editor fusion protein can comprise a Apolipoprotein B mRNA-editing complex (APOBEC) cytidine deaminase and the nucleotide mismatch is dC:A. The cell can be a T cell, Natural Killer (NK) cell, B cell, or CD34+ hematopoietic stem progenitor cell (HSPC).

In another aspect, provided herein is a method for producing a genetically modified cell. The method can comprise or consist essentially of: (a) introducing into a cell one or more plasmids, mRNAs, or proteins encoding: (i) a universal, precise staggered Cas9 editor comprising a nCas9 domain fused to MutY DNA glycosylase (MUTYH) and Apurinic Endonuclease 1 (APE1), wherein the nCas9 domain comprises a RuvC nuclease domain; (ii) a synthetic chimeric ssODN-ssORN duplex, wherein at least a portion of the ssORN is complementary to that of the Cas9 d-loop and comprises a 8-Oxoguanine (OG); and (ii) one or more gRNAs having complementarity to a target nucleic acid sequence to be genetically modified; and (b) culturing the introduced cell under conditions that promote modification of the target nucleic acid sequence targeted by the one or more gRNAs, whereby the target nucleic acid sequence is modified by the staggered Cas9 editor relative to unmodified cell, and whereby a genetically modified cell is produced. The universal, precise staggered Cas9 editor can comprise MUTYH-APE1-nCas9-PCV2. The cell can be a T cell, Natural Killer (NK) cell, B cell, or CD34+ hematopoietic stem progenitor cell (HSPC).

In a further aspect, provided herein is a genetically modified cell obtained according to a method of this disclosure.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention and to cover all modifications, equivalents and alternatives. Reference should therefore be made to the claims recited herein for interpreting the scope of the invention.

### BRIEF DESCRIPTION OF THE DRAWINGS

FIGS. 1A-1B demonstrate the formation of R-loop:RNA oligo DNA:RNA heteroduplex. (A) Schematic of DNA:RNA heteroduplex formation experiment. dCas9, a Cy3 labelled DNA and a FITC labelled oligonucleotide were combined. When annealing of the oligonucleotide to the ribonucleoprotein complex occurs, excitation of the FITC allows for FRET with the Cy3 fluorophore, emitting at 560 nm. (B) Oligonucleotides are able to hybridize to the R-loop of the RNP complex. In the presence of a complementary oligonucleotide FRET occurs, indicating hybridization of the oligonucleotide with the R-loop is occurring. When a non-matched sgRNA is used, no R-loop is formed and no FRET occurs, indicating the hybridization is specific. Salmon sperm (SS) DNA was also added to demonstrate that the FRET was specific to complementary oligonucleotides. Multiple lines indicate differing lengths of DNA including 45, 48, 51, 54, 57, and 60 bp in length.

FIGS. 2A-2C illustrate a base editing embodiment, including upABE construct and mechanism. A) Schematic of upABE protein construct consisting of a double-stranded nucleic acid adenosine deaminase domain, a peptide linker, the core Cas9 complex with a nicking mutation, and a single stranded nucleic acid binding domain such the HUH-endonuclease (His-U-His where U is a hydrophobic residue) PCV2 (Porcine Circovirus 2) Rep protein or HUH-endonuclease or nucleic acid binding domain. B) Schematic of ch-ssON single stranded nucleic acid binding domain linkage sequence, such as PCV2 Rep, variable linker of polynucleotides, single stranded nucleic acid, such as ssRNA that is complementary to the Cas9 R-loop with a mismatch to direct the site of editing. ch-ssON is covalently linked to upABE complex in 1:1 molar ratio at room temperature in Opti-MEM. C) Covalently linked complex binds target DNA, and forms a heteroduplex between the Cas9 R-loop and ch-ssON. Mismatch dictated by the ch-ssON directs the adenosine deaminase domain to the target base. Nicking of the antisense strand by the core Cas9 complex induces degradation of the non-edited strand and induces repair from the nascent inosine via MMR DNA polymerase. General construct design also applies to upBE and upCas9, per modifications specified in text.

FIGS. 3A-3C illustrate embodiments of ultraprecise base editing. (A) Schematic illustrates a VPg linked ssORN for precise base editing. Similar to the HUH-mediated tagging of the RNP complex, a homolog/paralog/analog of the MNV1 VPg protein is used to covalently tether a ssORN. MNV1 VPg covalently links to ssRNA based on a 5′-recognition sequence. Once tethered, base editing proceeds through a similar mechanism as the ch-ssORN HUH-endonuclease-mediated tethering (see FIG. 2C). (B) Schematic illustrates precise base editing using a 5′ extended sgRNA. The 5′ end of the sgRNA is extended to contain complementarity to the non R-loop strand. An A:C mismatch in the DNA:RNA heteroduplex is introduced via the 5′ extended sgRNA complex distal to the PAM. The deaminase is free then act on the mismatch to deaminate the inosine, resolving the mismatch. The core Cas9 complex comprises a single SpCas9(H480A) mutation which nicks the R-loop containing strand. Mismatch repair favors the degradation of the non-edited, nicked strand, thereby using the inosine as a template for DNA repair and replication allowing for propagation of the base edit. (C) Schematic illustrates precise base editing using a 3′ extended sgRNA in which the 3′ end of a sgRNA is extended to contain complementary sequence to the non R-loop strand. An A:C mismatch in the DNA:RNA heteroduplex with the R-loop is introduced via the 3′ extension of the sgRNA. The deaminase is free to act on the mismatch to deaminate the inosine, resolving the mismatch. The core Cas9 complex comprises a single SpCas9(D10A) mutation which nicks the non-edited, non-R-loop strand. Mismatch repair favors the degradation of the non-edited, nicked strand, thereby using the inosine as a template for DNA repair and replication allowing for propagation of the base edit.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The methods, systems, and compositions described herein are based at least in part on the inventors' development of highly precise base editors (also known as “nucleobase editors”). Generally, base editing is unlike CRISPR-based editing in that it does not cut double-stranded DNA. Instead, base editors use deaminase enzymes to precisely rearrange some of the atoms in one of the four bases that make up DNA or RNA, converting the base without altering the bases around it. First generation base editors are targeted to a specific locus by a guide RNA (gRNA), and they can convert cytidine to uridine within a small editing window near the protospacer adjacent motif (PAM) site. Uridine is subsequently converted to thymidine through base excision repair, creating a C->T change (or G->A on the opposite strand). Third-generation base editors (BE3 systems), in which base excision repair inhibitor UGI is fused to the Cas9 nickase, nick the unmodified DNA strand so that the cell is encouraged to use the edited strand as a template for mismatch repair. As a result, the cell repairs the DNA using a U-containing strand (introduced by cytidine deamination) as a template, copying the base edit. Fourth generation base editors (BE4 systems) employ two copies of base excision repair inhibitor UGI. Adenine base editors (ABEs) have been developed that efficiently convert targeted A·T base pairs to G·C (approximately 50% efficiency in human cells) in genomic DNA with high product purity (typically at least 99.9%) and low rates of indels (typically no more than 0.1%).

The inventors have improved upon existing base editors by developing universal, highly-precise adenosine deaminase base editors (upABE); universal, highly-precise cytidine deaminase base editors (upBEs); and universal, highly-precise staggered Cas9 nucleases (upCas9). As described herein, the improved base editors comprise a single-stranded oligonucleotide DNA (ssODN) or single-stranded oligonucleotide RNA (ssORN) binding domain, a core nCas9-gRNA complex and a deaminase (or nuclease) that edits mismatches in DNA:RNA heteroduplexes. As used herein, the term “nCas9” refers to a Cas9 enzyme variant that induces a single stranded break, as opposed to a double stranded break. Advantages of these methods, systems, and compositions are multifold and described herein. In particular, the advanced technology of this disclosure has immediate translational and commercial applications. For example, methods are useful for correcting disease-causing point mutations and generating novel cell products (e.g., engineered cell products) for therapeutic applications. The methods are particularly well-suited for improved methods of treating monogenic diseases such as sickle cell anemia, SCID-A, and β-thalasemia for which highly precise editing of aberrant nucleotides can restore normal cell function.

Accordingly, in a first aspect, provided herein is a universal, precise adenosine deaminase base editor (“upABE”) and methods of using the base editor complex with targeted dA:C mismatches for highly precise gene editing. Preferably, base editor complex comprising a variant of a dsRNA adenosine deaminase enzyme, ADAR1 and ADAR2. Variants having E->Q amino acid substitutions (“hADARd^{E>Q} variants”) such as, for example, hADAR1d^{E1008Q}, hADAR2d^{E488Q}, hADAR2d^{E428Q} are capable of selectively deaminating deoxyadenosine in dA:C mismatches within a DNA:RNA heteroduplex in vitro.¹⁶ Other variant ADAR proteins that can be used for the methods of this disclosure are described herein. Recently, researchers at the University of Minnesota described a Porcine Circovirus Rep protein (PCV2)-nCas9 fusion enzyme that can be recombinantly expressed and covalently linked to a ssODN homology directed repair (HDR) template in vitro for enhanced HDR rates in an immortalized cell line.¹⁵ In preferred embodiments, the hADARd^{E>Q}- is covalently linked to a nCas9-gRNA complex. In some embodiments, the universal, highly precise adenosine deaminase base editor is produced by fusing a variant of a dsRNA adenosine deaminase enzyme to an nCas9-PCV2-ch-ssON backbone. The resulting hADARd^{E>Q}-nCas9-PCV2 fusion enzyme forms a complex with a synthetic chimeric ssODN-ssORN (“ch-ssON”) by covalent linkage, where a portion of the ssORN is complementary to that of the Cas9 d-loop and comprises a “A” mismatch. In some cases, the fusion enzyme comprises hADAR1d^{E1008Q}-nCas9-PCV2. In other cases, the fusion enzyme comprises hADAR2d^{E488Q}-nCas9-PCV2 or hADAR2d^{E528Q}-nCas9-PCV2.

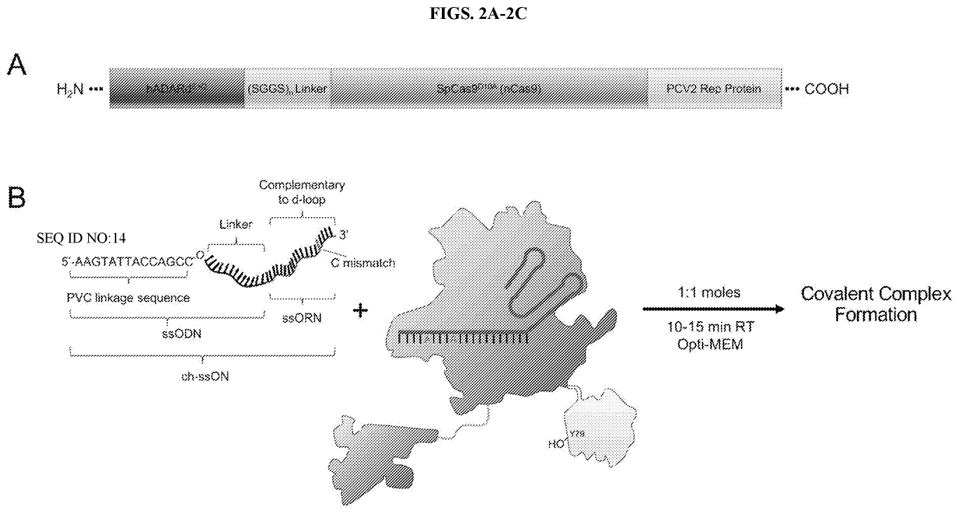

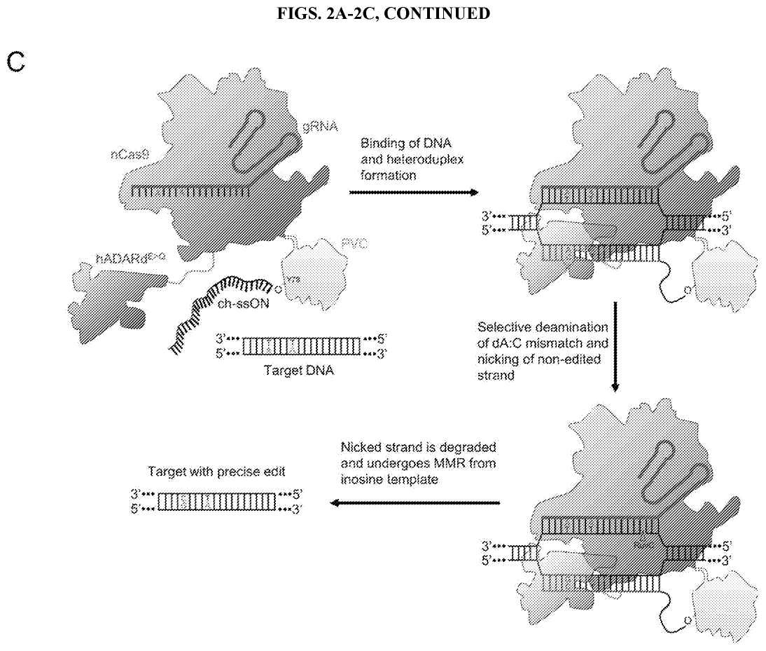

The gRNA directs the base editor complex to the target DNA sequence to which it is complementary, where the ssORN portion of the base editor complex forms a DNA:RNA heteroduplex with the target DNA. As used herein, the term “highly precise” refers to the ability of base editors of this disclosure to induce highly efficient and specific base editing with significantly reduced rates of indel formation relative to conventional base editors. With respect to upABE, highly precise base editing is achieved by the presence of a C mismatch in the complementary ssORN (see FIG. 2C). Without being bound to any particular mechanism or mode of action, deamination of the dA>dI will resolve the mismatch and inhibits further editing of any adjacent non-target adenosines, while nicking of the non-target strand by nCas9 would stimulate degradation of the non-edited strand. As such, mismatch repair is induced to repair the degraded strand using the nascent inosine as a template (FIG. 2C). In this manner, the base editors described herein present an unprecedented ability to precisely correct G:C>A:T mutations with virtually no unwanted indels.

In another aspect, provided herein is a universal, highly precise cytidine deaminase base editor (“upBE”) and methods of using the upBE complex with targeted mismatches for highly precise gene editing. Cytidine deaminase base editors have shown to be highly processive editors.^{10,18,19} In the context of base editing for the correction of pathogenic mutations, this is especially problematic due to the high rates on unwanted bystander mutations.²⁰ Apolipoprotein B mRNA-editing complex (APOBEC) cytidine deaminase allows for targeted gene disruption in which a single base substitution of thymidine in place of cytidine. Recently, the crystal structure of APOBEC3A bound to a ssDNA cytidine substrate was solved, which demonstrated a base flipping mechanism was required for the target cytidine to reach the active site.²¹ To mitigate bystander mutations, the cytidine deaminase base editors described herein are configured to selectively edit dC>dU at dC:A mismatches.

In preferred embodiments, the universal, highly precise cytidine deaminase base editor comprises a synthetic chimeric ssODN-ssORN (“ch-ssON”) that is covalently linked to a nCas9-gRNA complex, where a portion of the ssORN is complementary to that of the Cas9 d-loop and comprises a dC:A mismatch. Preferably, the gRNA is configured for hybridization to a target DNA sequence. Also covalently linked to the ch-ssON is an APOBEC-nCas9-PCV2 fusion enzyme. By covalently linking the fusion enzyme to a DNA:ssON heteroduplex in which the ssORN comprises a dC:A mismatch, target cytidines are selectively flipped out of the heteroduplex by the bulk mismatch and deaminated by the APOBEC. Similar to upABE, upon deamination of dC>dU, the nascent dU forms a dU:A Watson-Crick basepair with the ssON, thereby resolving the mismatch bubble and preventing further deamination of bystander cytidines. Referring to FIG. 2C, subsequent nicking of the non-target strand by nCas9 stimulates degradation of the non-edited strand, which induces mismatch repair to repair the degraded strand using the nascent uracil as a template.

In another aspect, provided herein is a universal, highly precise staggered Cas9 nuclease (upCas9) and methods of using the upCas9 with targeted mismatches for highly precise gene editing. Current methods for generating 5′ overhangs with Cas9 to preferentially mediate HDR rely on the use of a double nick strategy using nCas9 and two staggered gRNAs.^{6,7} While this approach can successfully target single sites, it has limited utility for multiplexed reactions, where multiple high-affinity gRNAs are required and the potential off-target effects is compounded. Furthermore, there has been considerable renewed concern about the potential off-target effects of full Cas9 nuclease activity at off-target sites in light of recent evidence demonstrating the large scale deletions and chromosomal rearrangements that can occur with Cas9 editing.²² As an improved alternative to the current Cas9 nuclease or the double nickase strategy, provided here is a universal, highly precise staggered Cas9 nuclease that generates a 5′ overhang cut and uses a programmable 8-Oxoguanine (OG) in the ch-ssON to direct the site of the secondary nick. In preferred embodiments, the universal, highly precise highly precise staggered Cas9 nuclease (upCas9) comprises a fusion enzyme comprising a MutY DNA glycosylase (MUTYH) and Apurinic Endonuclease 1 (APE1), whereby the resulting upCas9 comprises MUTYH-APE1-nCas9-PCV2. MutY DNA Glycosylase (MUTYH) is a human DNA glycosylase in the base excision repair pathway which hydrolyzes genomic adenosine from the deoxyribose across from the oxidized mutagenic guanine, 8-Oxoguanine (OG), thus generating an abasic site.^{23,24} Following hydrolysis, Apurinic Endonuclease 1 (APE1) binds to the abasic site and hydrolyzes the phosphate backbone of the abasic site at the 3′ hydroxyl of the immediately upstream base. Furthermore, MUTYH and APE1 are known to form an active complex with one another that coordinates the removal of OG and subsequent phosphate backbone cleavage.^{25,26} By fusing MUTYH and APE1 to form a single chimeric enzyme, the resulting enzyme possesses the dual function of adenosine excision and strand nicking across a dA:dOG mismatch.

In preferred embodiments, the universal, highly precise staggered Cas9 nuclease (upCas9) is produced by fusing the MUTYH-ABE fusion enzyme to an nCas9-ch-ssON backbone. If the ssON is configured to contain an oxidized mutagenic guanine across from an adenosine in the target R-loop, the upCas9 directs the dual glycosylase-endonuclease to create a single stranded nick in the target R-loop. Subsequently, the active RuvC nuclease domain of the nCas9 nicks the antisense target strand, thereby inducing a double stranded break (DSB) with 5′ overhangs. In this manner, the upCas9 is leveraged for homology directed repair of a target site without the need for multiple gRNAs. Furthermore, the necessity of an adenosine across the engineered OG in the ssON creates an additional specificity requirement for complete DSB induction. As a result, the upCas9 is less likely to have off-target effects.

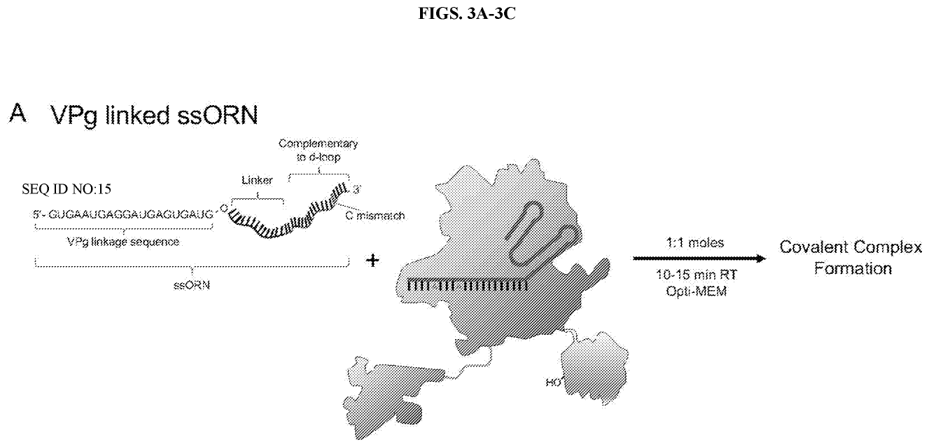

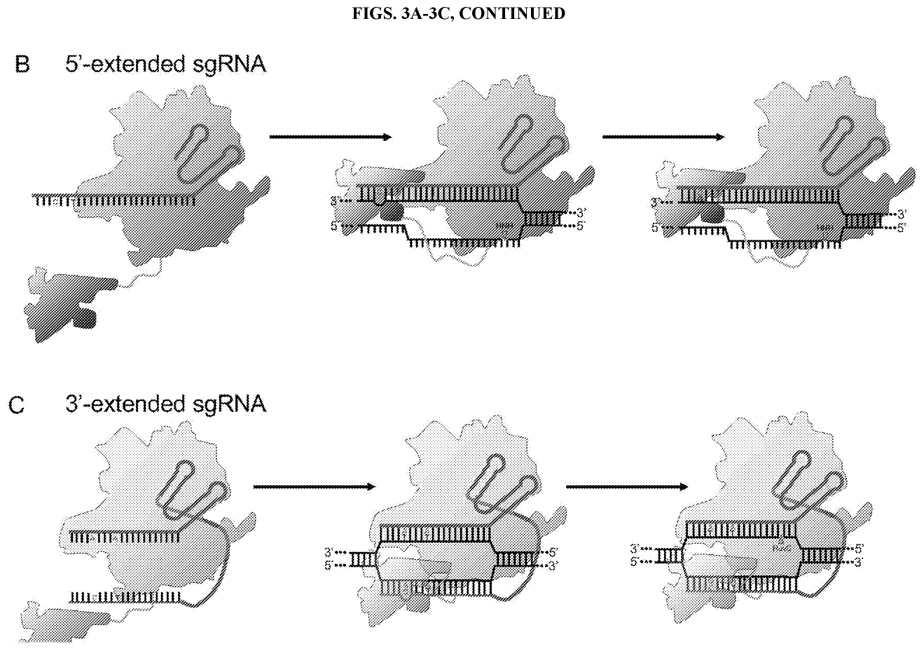

In some cases, a method of highly precise base editing of this disclosure comprises alternative means of forming a heteroduplex with a single stranded oligonucleotide comprising a base mismatch. For example, in one embodiment, a homolog (or paralog or analog) of the murine norovirus 1 (MNV1) VPg protein can bind covalently a ssORN based on a 5′ recognition sequence. This embodiment is depicted in FIG. 3A. Once tethered, base editing proceeds through a similar mechanism as the ch-ssORN HUH-mediated tethering. Sequences of exemplary VPg orthologs and their recognition sequences are set forth in Table 1.

In another embodiment, depicted in FIG. 3B, precise base editing employs a 5′ extended sgRNA. The 5′ end of the sgRNA is extended to contain complementarity to the non R-loop strand. An A:C mismatch in the DNA:RNA heteroduplex is introduced via the 5′ extended sgRNA complex distal to the PAM. The deaminase is free then act on the mismatch to deaminate the inosine, resolving the mismatch. The core Cas9 complex comprises a single SpCas9(H480A) mutation which nicks the R-loop containing strand. Mismatch repair favors the degradation of the non-edited, nicked strand, thereby using the inosine as a template for DNA repair and replication allowing for propagation of the base edit.

In another embodiment, depicted in FIG. 3C, precise base editing employs a 3′ extended sgRNA. The 3′ end of the sgRNA is extended to contain complementary sequence to the non R-loop strand. An A:C mismatch in the DNA:RNA heteroduplex with the R-loop is introduced via the 3′ extension of the sgRNA. The deaminase is free to act on the mismatch to deaminate the inosine, resolving the mismatch. The core Cas9 complex comprises a single SpCas9(D10A) mutation which nicks the non-edited, non-R-loop strand. Mismatch repair favors the degradation of the non-edited, nicked strand, thereby using the inosine as a template for DNA repair and replication allowing for propagation of the base edit.

Any Cas enzyme can be used according to the methods and systems of this disclosure. The terms “Cas” and “CRISPR-associated Cas” are used interchangeably herein. The Cas enzyme can be any naturally-occurring nuclease as well as any chimeras, mutants, homologs, or orthologs. In some embodiments, one or more elements of a CRISPR system is derived from a particular organism comprising an endogenous CRISPR system, such as Streptococcus pyogenes (SP) CRISPR systems or Staphylococcus aureus (SA) CRISPR systems. The CRISPR system is a type II CRISPR system and the Cas enzyme is Cas9 or a catalytically inactive Cas9 (dCas9). Other non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. A comprehensive review of the Cas protein family is presented in Haft et al. (2005) Computational Biology, PLoS Comput. Biol. 1:e60. At least 41 CRISPR-associated (Cas) gene families have been described to date.

Any suitable means of nucleic acid construct delivery can be used to introduce nucleic acids encoding the base editors or components thereof into a cell. For example, the ssODN, ssORN, or the synthetic chimeric single-stranded oligonucleotide complex (ch-ssON) can be expressed from a plasmid or a viral vector, or is delivered to a cell as an RNA. In some cases, the base editor enzyme is expressed from a plasmid or a viral vector, or is delivered to a cell as an RNA. In other cases, the base editor enzyme is delivered to cell as a protein (e.g., a recombinantly expressed protein). As used herein, the term “vector” is intended to mean a nucleic acid molecule capable of transporting another nucleic acid. By way of example, a vector which can be used in the present invention includes, but is not limited to, a viral vector (e.g., retrovirus, adenovirus, baculovirus), a plasmid, a RNA vector or a linear or circular DNA or RNA molecule which may consist of a chromosomal, non-chromosomal, semi-synthetic or synthetic nucleic acid. Large numbers of suitable vectors are known to those of skill in the art and commercially available. Preferred vectors are those capable of autonomous replication (episomal vector) and/or expression of nucleic acids to which they are operably linked (expression vectors). In some embodiments, the linkage between the core enzyme complex and the ch-ssON will occur intracellularly or in the extracellular space of an organism.

It will be understood that fusion enzymes of the programmable base editors and nucleases of the invention can be modified relative to the enzymes exemplified in this disclosure, for example, in order to tailor a programmable base editor or nuclease for a particular application. For example, in some embodiments, the protein construct can comprise a homolog or ortholog of a particular enzyme (e.g., homolog or ortholog of a Cas nuclease, hADARd^{E>Q}, APOBEC cytidine deaminase, MutY DNA glycosylase, or apurinic endonuclease). Homologs and orthologs include, without limitation, Streptococcus pyogenes Cas9, Staphylococcus aureus Cas9, Campylobacter jejuni Cas9, Lachnospiraceae bacterium Cpf1, Neisseria meningitidis Cas9, Streptococcus thermophilus Cas9, or any engineered or mutated Cas9 variant; ADAR1, ADAR2, ADAR3/RED2, ADAT1, ADAT2, ADAT3, ADARB1. APOBEC: APOBEC1, APOBEC2, APOBEC3A, APOBEC3B, APOBEC3C, APOBEC3E, APOBEC3F, APOBEC3G, APOBEC3H, APOBEC4, AID, rat APOBEC1, sea lamprey AI; HUH-endonuclease from Porcine circovirus 2 (PCV2), duck circovirus (DCV), fava bean necrosis yellow virus (FBNYV), Streptococcus agalactiae replication protein (RepB), Fructobacillus tropaeoli RepB, Escherichia coli conjugation protein TraI, Escherichia coli mobilization protein A, Staphylococcus aureus nicking enzyme (NES); VPg proteins from Norovirus, Vesivirus, Sapovirus, Lagovirus, Recovirus, Nebovrius, Homo sapiens MUTYH, Mus musculus Mutyh, Rattus norvegicus Mutyh, Pan-troglodytes MUTYH, Escherichia coli mutY, Bacillus subtilis mutY, Arabidiosus thaliana MYH; Saccharomyces cerevisiae APE1, Arabidopsis thaliana APE1L, Caenorhabditis elegans ape-1, Homo sapiens NTHL1, Homo sapiens APE2. While these enzymes are exemplary of suitable base editors and nucleases for use in the disclosed systems and methods a skilled artisan will recognize a range of base editors and nucleases are suitable for use, and a skilled artisan will know how to appropriately select a suitable base editor or nuclease.

In some cases, the protein construct comprises one or more variations (e.g., mutation, insertion, deletion, truncation) or comprises a functionally equivalent protein in place of a Cas nuclease, hADARd^{E>Q}, APOBEC cytidine deaminase, MutY DNA Glycosylase, or APE. In some cases, the protein construct is modified to comprise a different single-stranded RNA binding domain or different single-stranded DNA binding domain.

In some cases, the dsRNA adenosine deaminase (also known as double-stranded RNA-specific adenosine deaminase) comprises an amino acid substitution of an E to a Q at position 1008, as numbered relative to Homo sapiens (Human) ADAR (Uniport P55265):

(SEQ ID NO: 1)
MNPRQGYSLSGYYTHPFQGYEHRQLRYQQPGPGSSPSSFLLKQIEFLKG
QLPEAPVIGKQTPSLPPSLPGLRPREPVLLASSTRGRQVDIRGVPRGVH
LRSQGLQRGFQHPSPRGRSLPQRGVDCLSSHFQELSIYQDQEQRILKFL
EELGEGKATTAHDLSGKLGTPKKEINRVLYSLAKKGKLQKEAGTPPLWK
IAVSTQAWNQHSGVVRPDGHSQGAPNSDPSLEPEDRNSTSVSEDLLEPF
IAVSAQAWNQHSGVVRPDSHSQGSPNSDPGLEPEDSNSTSALEDPLEFL
DMAEIKEKICDYLFNVSDSSALNLAKNIGLTKARDINAVLIDMERQGDV
YRQGTTPPIWHLTDKKRERMQIKRNTNSVPETAPAAIPETKRNAEFLTC
NIPTSNASNNMVTTEKVENGQEPVIKLENRQEARPEPARLKPPVHYNGP
SKAGYVDFENGQWATDDIPDDLNSIRAAPGEFRAIMEMPSFYSHGLPRC
SPYKKLTECQLKNPISGLLEYAQFASQTCEFNMIEQSGPPHEPRFKFQV
VINGREFPPAEAGSKKVAKQDAAMKAMTILLEEAKAKDSGKSEESSHYS
TEKESEKTAESQTPTPSATSFFSGKSPVTTLLECMHKLGNSCEFRLLSK
EGPAHEPKFQYCVAVGAQTFPSVSAPSKKVAKQMAAEEAMKALHGEATN
SMASDNQPEGMISESLDNLESMMPNKVRKIGELVRYLNTNPVGGLLEYA
RSHGFAAEFKLVDQSGPPHEPKFVYQAKVGGRWFPAVCAHSKKQGKQEA
ADAALRVLIGENEKAERMGFTEVTPVTGASLRRTMLLLSRSPEAQPKTL
PLTGSTFHDQIAMLSHRCFNTLTNSFQPSLLGRKILAAIIMKKDSEDMG
VVVSLGTGNRCVKGDSLSLKGETVNDCHAEIISRRGFIRFLYSELMKYN
SQTAKDSIFEPAKGGEKLQIKKTVSFHLYISTAPCGDGALFDKSCSDRA
MESTESRHYPVFENPKQGKLRTKVENGEGTIPVESSDIVPTWDGIRLGE
RLRTMSCSDKILRWNVLGLQGALLTHFLQPIYLKSVTLGYLFSQGHLTR
AICCRVTRDGSAFEDGLRHPFIVNHPKVGRVSIYDSKRQSGKTKETSVN
WCLADGYDLEILDGTRGTVDGPRNELSRVSKKNIFLLFKKLCSFRYRRD
LLRLSYGEAKKAARDYETAKNYFKKGLKDMGYGNWISKPQEEKNFYLCP
V.

In some cases, the dsRNA adenosine deaminase (also known as double-stranded RNA-specific editase 1) comprises an amino acid substitution of an E to a Q at position 488, as numbered relative to Homo sapiens (Human) ADARB1/ADAR2 (Uniprot ID P78563):

(SEQ ID NO: 2)
MDIEDEENMSSSSTDVKENRNLDNVSPKDGSTPGPGEGSQLSNGGGGGP
GRKRPLEEGSNGHSKYRLKKRRKTPGPVLPKNALMQLNEIKPGLQYTLL
SQTGPVHAPLFVMSVEVNGQVFEGSGPTKKKAKLHAAEKALRSFVQFPN
ASEAHLAMGRTLSVNTDFTSDQADFPDTLFNGFETPDKAEPPFYVGSNG
DDSFSSSGDLSLSASPVPASLAQPPLPVLPPFPPPSGKNPVMILNELRP
GLKYDFLSESGESHAKSFVMSVVVDGQFFEGSGRNKKLAKARAAQSALA
AIFNLHLDQTPSRQPIPSEGLQLHLPQVLADAVSRLVLGKFGDLTDNFS
SPHARRKVLAGVVIVITTGTDVKDAKVISVSTGTKCINGEYMSDRGLAL
NDCHAEIISRRSLLRFLYTQLELYLNNKDDQKRSIFQKSERGGFRLKEN
VQFHLYISTSPCGDARIFSPHEPILEGSRSYTQAGVQWCNHGSLQPRPP
GLLSDPSTSTFQGAGTTEPADRHPNRKARGQLRTKIESGEGTIPVRSNA
SIQTWDGVLQGERLLTMSCSDKIARWNVVGIQGSLLSIFVEPIYFSSII
LGSLYHGDHLSRAMYQRISNIEDLPPLYTLNKPLLSGISNAEARQPGKA
PNFSVNWTVGDSAIEVINATTGKDELGRASRLCKHALYCRWMRVHGKVP
SHLLRSKITKPNVYHESKLAAKEYQAAKARLFTAFIKAGLGAWVEKPTE
QDQFSLTP.

Other ADAR1 or ADAR2 isoforms comprising other amino acid substitutions may be used. For example, the variant ADAR2 can be ADAR2^{E528Q} having the following amino acid sequence:

(SEQ ID NO: 3)
MDIEDEENMSSSSTDVKENRNLDNVSPKDGSTPGPGEGSQLSNGGGGGP
GRKRPLEEGSNGHSKYRLKKRRKTPGPVLPKNALMQLNEIKPGLQYTLL
SQTGPVHAPLFVMSVEVNGQVFEGSGPTKKKAKLHAAEKALRSFVQFPN
ASEAHLAMGRTLSVNTDFTSDQADFPDTLFNGFETPDKAEPPFYVGSNG
DDSFSSSGDLSLSASPVPASLAQPPLPVLPPFPPPSGKNPVMILNELRP
GLKYDFLSESGESHAKSFVMSVVVDGQFFEGSGRNKKLAKARAAQSALA
AIFNLHLDQTPSRQPIPSEGLQLHLPQVLADAVSRLVLGKFGDLTDNFS
SPHARRKVLAGVVIVITTGTDVKDAKVISVSTGTKCINGEYMSDRGLAL
NDCHAEIISRRSLLRFLYTQLELYLNNKDDQKRSIFQKSERGGFRLKEN
VQFHLYISTSPCGDARIFSPHEPILEGSRSYTQAGVQWCNHGSLQPRPP
GLLSDPSTSTFQGAGTTEPADRHPNRKARGQLRTKIESGQGTIPVRSNA
SIQTWDGVLQGERLLTMSCSDKIARWNVVGIQGSLLSIFVEPIYFSSII
LGSLYHGDHLSRAMYQRISNIEDLPPLYTLNKPLLSGISNAEARQPGKA
PNFSVNWTVGDSAIEVINATTGKDELGRASRLCKHALYCRWMRVHGKVP
SHLLRSKITKPNVYHESKLAAKEYQAAKARLFTAFIKAGLGAWVEKPTE
QDQFSLTP.

Although constructs encoding human proteins are described herein, those of skill in the art will appreciate that non-human and/or synthetic amino acid sequences can be used in place of human amino acid sequences. It will also be appreciated that amino acid analogs can be inserted or substituted in place of naturally occurring amino acid residues. As used herein, the term “amino acid analog” refers to amino acid-like compounds that are similar in structure and/or overall shape to one or more of the twenty L-amino acids commonly found in naturally occurring proteins. Amino acid analogs are either naturally occurring or non-naturally occurring (e.g. synthesized). If an amino acid analog is incorporated by substituting natural amino acids, any of the 20 amino acids commonly found in naturally occurring proteins may be replaced. While amino acids can be replaced (substituted) with amino acid analogs, in some cases amino acid analogs are inserted into a protein. For example, a codon encoding an amino acid analog can be inserted into the polynucleotide encoding the protein.

Any appropriate linker peptide can be used to bridge polypeptide constituents that comprise a fusion enzyme of this disclosure. As used herein, a “peptide linker” or “linker” is a polypeptide typically ranging from about 2 to about 50 amino acids in length, which is designed to facilitate the functional connection of two polypeptides into a linked fusion polypeptide. The term functional connection denotes a connection that facilitates proper folding of the polypeptides into a three dimensional structure that allows the linked fusion polypeptide to mimic some or all of the functional aspects or biological activities of the proteins from which its polypeptide constituents are derived. The term functional connection also denotes a connection that confers a degree of stability required for the resulting linked fusion polypeptide to function as desired. In each particular case, the preferred linker length will depend upon the nature of the polypeptides to be linked and the desired activity of the linked fusion polypeptide resulting from the linkage. Generally, the linker should be long enough to allow the resulting linked fusion polypeptide to properly fold into a conformation providing the desired biological activity.

In some embodiments, it may be advantageous to arrange protein constructs in alternative orders. In some embodiments, it may also be advantageous to combine facets of the programmable base editors and nucleases of this disclosure to obtain different constructs. For example, certain components of upABE, upBE, and/or upCas9 may be combined to form a new protein construct.

In some embodiments, nucleic acids in either the gRNA or ssON are ribonucleotides or deoxynucleotides.

In some embodiments, the nucleotides are of a non-canonical (such as pseudouridyl, 8-oxoguanine, 6-methyl adenine) or of synthetic identity (such as 8-thioguanine, diamino purine, isocystine).

In some embodiments, linking bonds between the nucleotides are modified such as via a phosphorthioate bond.

In some embodiments, the substitution of the ribose are modified, such as 2′ fluorines on the sugar, or other modified sugars.

In some embodiments, a nucleic acid of a construct described herein comprises one or more chemical modifications. In some cases, the nucleic acid is tagged such as with a fluorophore.

In some embodiments, the nucleic acid will be conjugated to the protein in a different manner.

In some cases, the guide RNA molecule (gRNA) is expressed from a plasmid or a viral vector, or is delivered to a cell as an RNA. Generally, a gRNA comprises a nucleotide sequence that is partially or wholly complementary a target sequence in the genome of a cell (“a gRNA target site”) and comprises a target base pair. A gRNA target site also comprises a Protospacer Adjacent Motif (PAM) located immediately downstream from the target site. Examples of PAM sequence are known (see, e.g., Shah et al., RNA Biology 10 (5): 891-899, 2013). For some embodiments, the gRNA preferably comprises a sequence of at least 10 contiguous nucleotides, and often a sequence of 18-22 contiguous nucleotides or more. In some embodiments, a guide RNA molecule can be from 20 to 300 or more bases in length, or more. In certain embodiments, a guide RNA molecule can be from 20 to 300 bases in length, or 20 to 120 bases, or 30 to 50 bases, or 39 to 46 bases. As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

In some cases, it is advantageous to use chemically modified gRNAs having increased stability when transfected into mammalian cells. For example, gRNAs can be chemically modified to comprise 2′-O-methyl phosphorthioate modifications on at least one 5′ nucleotide and at least one 3′ nucleotide of each gRNA. In some cases, the three terminal 5′ nucleotides and three terminal 3′ nucleotides are chemically modified to comprise 2′-O-methyl phosphorthioate modifications.

In some embodiments, the gRNA is covalently bound to the Cas9 complex via a VPg protein for the purpose of effective transport of the gRNA and Cas9 to an organelle including, but not limited to, a mitochondria or chloroplast. Provided herein are also methods for genome engineering (e.g., for altering or manipulating the expression of one or more genes or one or more gene products) in prokaryotic or eukaryotic cells, in vitro, in vivo, or ex vivo. In particular, the methods provided herein are useful for targeted base editing or base correction in any animal, plant, or prokaryotic cell. In some cases, the cell is a mammalian cell. Mammalian cells include, without limitation, human T cells, natural killer (NK) cells, CD34+ hematopoietic stem progenitor cells (HSPCs) (e.g., umbilical cord blood HSPCs), and fibroblasts (e.g., MPS1 fibroblasts, Fanconi Anemia fibroblasts), terminally differentiated cells, multipotent stem cells, and pluripotent stem cells. It was previously shown that fibroblasts derived from a Fanconi Anemia patient and, therefore, DNA repair deficient are still amenable to base editing. Accordingly, also provided herein are genetically engineered cells that have been modified according to these methods.

As used herein, the terms “genetically modified” and “genetically engineered” are used interchangeably and refer to a prokaryotic or eukaryotic cell that includes an exogenous polynucleotide, regardless of the method used for insertion. In some cases, the effector cell has been modified to comprise a non-naturally occurring nucleic acid molecule that has been created or modified by the hand of man (e.g., using recombinant DNA technology) or is derived from such a molecule (e.g., by transcription, translation, etc.). An effector cell that contains an exogenous, recombinant, synthetic, and/or otherwise modified polynucleotide is considered to be an engineered cell.

In some cases, a universal precise base editor construct is introduced into a cell to base editing correction of a pathogenic mutation in a target gene. The target sequence can be any disease-associated polynucleotide or gene, as have been established in the art. Examples of useful applications of mutation or ‘correction’ of an endogenous gene sequence include alterations of disease-associated gene mutations, alternations in sequence adjacent to a disease-associated gene, alterations in sequences encoding splice sites, alterations in regulatory sequences, alterations in sequences to cause a gain-of-function mutation, and/or alterations in sequences to cause a loss-of-function mutation, and targeted alterations of sequences encoding structural characteristics of a protein. In particular, universal precise base editors of this disclosure may be used to treat a monogenic disorder, which is a disease caused by mutation in a single gene. The mutation may be present on one or both chromosomes (one chromosome inherited from each parent). Examples of monogenic disorders include, without limitation, sickle cell disease, X-linked SCID (severe combined immune deficiency), Fanconi Anemia, β-thalasemia, cystic fibrosis, hemophilia, polycystic kidney disease, Huntington's Disease, Mucopolysaccharidosis, and Tay-Sachs disease.

In some embodiments, a universal precise base editor construct is configured to target a gene selected from the group consisting of HBB, HBG1, HBG2, HBA, COL7A1, ADA, CFTR, MPS, IDUA, IDS, SGSH, SGSH, NAGLU, HGSNAT, GSN, GALNS, GLB1, ARSB, GUSB, HYAL1, FCGR3A, PDCD1, TRAC TRBQ CISH, CTLA4, DCLREC, FANCA, FANCC, FANCD1, FANCD2, FANCF, COL7A1, TGFBR, CD247, CD3G, CD3D, and CD3E.

In some cases, a universal precise base editor construct (e.g., upABE, upBE, upCas9) is introduced into a cell to mediate the insertion of a chimeric antigen receptor (CAR) and/or T cell receptor (TCR), whereby the modified cell expresses the CAR and/or TCR. As used herein, the term “chimeric antigen receptor (CAR)” (also known in the art as chimeric receptors and chimeric immune receptors) refers to an artificially constructed hybrid protein or polypeptide comprising an extracellular antigen binding domains of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, a T cell can be engineered to express a CAR specific for molecule expressed on the surface of a particular cell (e.g., a tumor cell, B-cell lymphoma). For allogenic antitumor cell therapeutics not limited by donor-matching, it may be advantageous to use the constructs and methods described herein to insert nucleic acids encoding a CAR or TCR, but also to modify genes responsible for donor matching (TCR and HLA markers).

In other cases, a universal precise base editor construct can be used to mediate the insertion of an engineered immunoglobulin H (IgH), whereby the modified cell expresses IgH.

The universal precise base editor constructs (e.g., upABE, upBE, upCas9) provided herein are suitable for a wide variety of practical applications including medical, agricultural, commercial, education, and research purposes. Those of skill in the art will appreciate that selection of a universal precise base editor and the cell type in which gene editing shall occur will vary depending on the intended application. Depending on the application, programmable base editors of this disclosure can be introduced into pluripotent stem cells (e.g., embryonic stem cells, induced pluripotent stem cell), multipotent stem cells (e.g., hematopoietic stem cells, mesenchymal stem cells), somatic cells, or immune cells (e.g., T-cells, B-cells, monocytes, NK cells, CD34⁺ cells).

A base editing system as described herein may be introduced into a biological system (e.g., a virus, prokaryotic or eukaryotic cell, zygote, embryo, plant, or animal, e.g., non-human animal). A prokaryotic cell may be a bacterial cell. A eukaryotic cell may be, e.g., a fungal (e.g., yeast), invertebrate (e.g., insect, worm), plant, vertebrate (e.g., mammalian, avian) cell. A mammalian cell may be, e.g., a mouse, rat, non-human primate, or human cell. A cell may be of any type, tissue layer, tissue, or organ of origin. In some embodiments a cell may be, e.g., an immune system cell such as a lymphocyte or macrophage, a fibroblast, a muscle cell, a fat cell, an epithelial cell, or an endothelial cell. A cell may be a member of a cell line, which may be an immortalized mammalian cell line capable of proliferating indefinitely in culture.

In some embodiments, components of a construct described herein can be delivered to a cell in vitro, ex vivo, or in vivo. In some cases, a viral or plasmid vector system is employed for delivery of base editing components described herein. Preferably, the vector is a viral vector, such as a lenti- or baculo- or preferably adeno-viral/adeno-associated viral (AAV) vectors, but other means of delivery are known (such as yeast systems, microvesicles, gene guns/means of attaching vectors to gold nanoparticles) and are contemplated. In certain embodiments, nucleic acids encoding gRNAs and base editor fusion proteins are packaged for delivery to a cell in one or more viral delivery vectors. Suitable viral delivery vectors include, without limitation, adeno-viral/adeno-associated viral (AAV) vectors, lentiviral vectors. In some cases, non-viral transfer methods as are known in the art can be used to introduce nucleic acids or proteins in mammalian cells. Nucleic acids and proteins can be delivered with a pharmaceutically acceptable vehicle, or for example, encapsulated in a liposome. Other means of delivery are known (such as yeast systems, microvesicles, gene guns/means of attaching vectors to gold nanoparticles) and are contemplated. In some cases, cells are electroporated for uptake of gRNA and base editor (e.g., upABE, upBE, upCas9). In some cases, DNA donor template is delivered as Adeno-Associated Virus Type 6 (AAV6) vector by addition of viral supernatant to culture medium after introduction of the gRNA, base editor, and vector by electroporation.

Rates of insertion or deletion (indel) formation can be determined by an appropriate method. For example, Sanger sequencing or next generation sequencing (NGS) can be used to detect rates of indel formation. Preferably, the contacting results in less than 20% off-target indel formation upon base editing. The contacting results in a ratio of at least 2:1 intended to unintended product upon base editing.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids and/or other constructs of the invention may be isolated. As used herein, “isolated” means to separate from at least some of the components with which it is usually associated whether it is derived from a naturally occurring source or made synthetically, in whole or in part.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

So that the compositions and methods provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used herein and in the claims, the singular forms “a,” “an,” and “the” include the singular and the plural reference unless the context clearly indicates otherwise. Thus, for example, a reference to “an agent” includes a single agent and a plurality of such agents. Any reference to “or” herein is intended to encompass “and/or” unless otherwise stated.

Various exemplary embodiments of compositions and methods according to this invention in addition to those shown and described herein will become apparent to those skilled in the art from the foregoing description and the following examples and fall within the scope of the appended claims. Those skilled in the art will recognize, or be able to ascertain using no more than routine experimentation, many equivalents to the specific embodiments of the invention described herein. Such equivalents are intended to be encompassed by the following claims.

### Example 1

This example describes embodiments for ultraprecise base editing. Unlike conventional base editing methods, the presently described embodiments exploit the physiochemical properties and selectivity that can be conferred from a DNA:RNA heteroduplex in order to induce chemical changes to bases within the DNA:RNA heteroduplex. Rather than using the DNA:RNA heteroduplex as a starting point for generation of a new DNA molecule by reverse transcriptase to be incorporated into the genome, the inventors' technology employs direct modification of bases within the DNA:RNA heteroduplex.

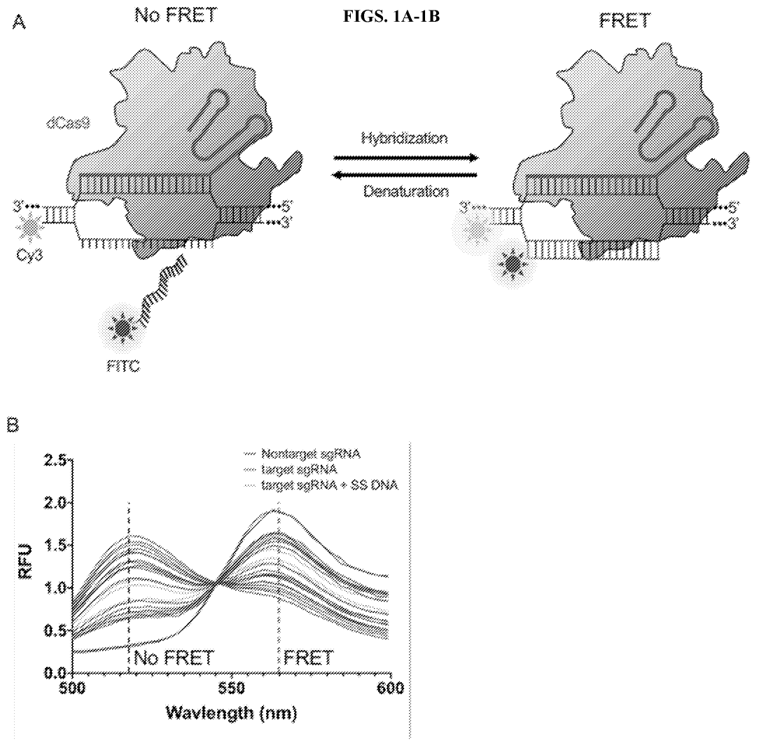

FIG. 1A shows a schematic of the DNA:RNA heteroduplex formation experiment. dCas9, a Cy3 labelled DNA and a FITC labelled oligonucleotide were combined. When annealing of the oligonucleotide to the ribonucleoprotein complex occurs, excitation of the FITC allows for FRET with the Cy3 fluorophore, emitting at 560 nm. As shown in FIG. 1, oligonucleotides are able to hybridize to the R-loop of the RNP complex. In the presence of a complementary oligonucleotide FRET occurs, indicating hybridization of the oligonucleotide with the R-loop is occurring. When a non-matched sgRNA is used, no R-loop is formed and no FRET occurs, indicating the hybridization is specific. Salmon sperm (SS) DNA was also added to demonstrate that the FRET was specific to complementary oligonucleotides. Multiple lines indicate differing lengths of DNA including 45, 48, 51, 54, 57, and 60 bp in length. Recombinantly expressed dCas9 protein, sgRNA, target Cy3-labelled-dsDNA, and FITC-labelled-oligonucleotide were combined in a 96-well plate and incubated for 1 hr at 25° C. The plate was analyzed in a plate reader using a 495 nm excitation, and emission was measured from 500 nm-600 nm. Emission signal was normalized across conditions with the emission value at 545 nm. These results demonstrate that a DNA:RNA heteroduplex forms between the R-loop and a oligonucleotide. Because the DNA:RNA heteroduplex forms, an A:C mismatch can also be introduced into this heteroduplex. Given the presence an adenosine deaminase that can act on A:C mismatches, this DNA:RNA heteroduplex will allow for efficient and precise editing of the target adenosine. Furthermore, this principle could be conferred to any potential mismatch induced into the heteroduplex that could be leveraged to direct an enzyme to perform any selective modification as described in this patent.

As shown in FIG. 3A, precise base editing can employ a VPg-linked single stranded RNA oligonucleotide (ssORN). Similar to the HUH-mediated tagging of the RNP complex described herein and illustrated in FIGS. 2A-2C, a homolog (or paralog or analog) of the murine norovirus 1 (MNV1) VPg protein covalently tethers a ssORN based on a 5′ recognition sequence. Covalent protein-RNA linkages to MNV1 VPg orthologs are described by, for example, Olspert et al. (PeerJ. 2016; 4: e2134). Once tethered, base editing proceeds through a similar mechanism as the ch-ssORN HUH-mediated tethering illustrated in FIG. 2C. Sequences of exemplary VPg orthologs and their recognition sequences are set forth in Table 1.

As shown in FIG. 3B, an alternative embodiment of precise base editing employs a 5′ extended sgRNA. The 5′ end of the sgRNA is extended to contain complementarity to the non R-loop strand. An A:C mismatch in the DNA:RNA heteroduplex is introduced via the 5′ extended sgRNA complex distal to the PAM. The deaminase is free to act on the mismatch to deaminate the inosine, thus resolving the mismatch. The core Cas9 complex comprises a single SpCas9(H480A) mutation which nicks the R-loop containing strand. Mismatch repair favors the degradation of the non-edited, nicked strand, thereby using the inosine as a template for DNA repair within the DNA:RNA heteroduplex and replication, allowing for propagation of the base edit. Binding of ABE to 5′ extended gRNA is demonstrated by Ryu et al. (Nature Biotechnology 2018, 36:536-539) for application of ABE-mediated adenine-to-guanine (A-to-G) single-nucleotide substitutions in a guide RNA (gRNA)-dependent manner in mouse embryos and adult mice.

As shown in FIG. 3C, an alternative embodiment of precise base editing employs a 3′ extended sgRNA. The 3′ end of the sgRNA is extended to contain complementary sequence to the non R-loop strand. An A:C mismatch in the DNA:RNA heteroduplex with the R-loop is introduced via the 3′ extension of the sgRNA. The deaminase is free to act on the mismatch to deaminate the inosine, resolving the mismatch. The core Cas9 complex comprises a single SpCas9(D10A) mutation which nicks the non-edited, non-R-loop strand. Mismatch repair favors the degradation of the non-edited, nicked strand, thereby using the inosine as a template for DNA repair and replication allowing for propagation of the base edit. Evidence that a 3′ extended sgRNA can form a DNA:RNA heteroduplex has been demonstrated by others. See Anzalone et al., Nature (2019).

Rather than using the DNA:RNA heteroduplex as a starting point for generation of a new DNA molecule by reverse transcriptase to be incorporated into the genome, the inventors' methods provided in this disclosure employ direct modification of bases within the DNA:RNA heteroduplex.

TABLE 1
VPg Binding Sequences

>MNV
(SEQ ID NO: 4)
GTGAATGAGGATGAGTGATG
>MF416380.1 Murine norovirus isolate MNV/NYC/Manhattan/poolF4, partial
genome
(SEQ ID NO: 5)
GTGAAATGAGGATGGCAACGCCATCTTCTGCGCCCTCTGTGCGCAACACAGAGAAACGCAAAAACAAAAA
GRCTTCATCTAARGCTAGYGTCTCCTTYGGAGCACCTAGCCTTCTCTCTTCGGAGAGTGAAGATGAAGTT
MAYTAYATGACCCCTCCTGAGCAGGAAGCTCAGCCCGGCRCCCTCGCGGCCCTTCATGCTGATGGGCCGC
ACGCCGGGCTCCCCGTGACGCGAAGTGATGCACGCGTGCTGATCTTCAATGAGTGGGAGGAGAGGAAGAA
GTCCGAGCCGTGGCTACGGCTGGACATGTCTGACAAGGCCATCTTCCGCCGCTACCCTCATCTGCGRCCT
AAGGAAGACAAGGCYGATGCGCCCTCCYATGCGGAGGACGCCATGGATGCAAGGGAGCCYGTGGTGGGRT
CCATYCTTGAGCAGGATGACCAYAAGTTCTACCACTACTCTGTCTACATCGGCAACGGTATGGTGATGGG
TGTCAACAACCCCGGCGCCGCCGTTTGCCAGGCTGTGATTGATGTGGARAAGCTCCACCTTTGGTGGAGG
CCAGTYTGGGAACCTCGCCAACCYCTCGACCCGGCTGAGTTGAGGAAGTGTGTYGGCATGACCGTCCCYT
ACGTGGCCACCACTGTCAATTGCTACCAGGTCTGCTGCTGGATTGTTGGGATCAAGGACACCTGGCTGAA
GAGRGCGAAGATATCCAGAGATTCGCCCTTCTACAGCCCYGTCCAGGACTGGAACATTGATCCCCAGGAG
CCCTTCATCCCGTCCAAGCTCAGGATGGTTTCTGATGGCATCYTAGTGGCTCTCTCAACGGTGATTGGTC
GGCCGATCAAGAACCTGCTGGCATCMGTGAAGCCGCTCAACATTCTGAACATCGTGTTGAGYTGTGACTG
GACTTTCTCGGGCATAGTCAACGCCCTGATCCTCCTTGCTGAGCTATTTGACATCTTTTGGACTCCCCCT
GATGTCACCAACTGGATGATCTCCATCTTTGGGGAATGGCAAGCCGAGGGGCCCTTCGACCTTGCCCTGG
ACGTTGTGCCCACCCTGCTTGGTGGGATTGGCATGGCCTTCGGCCTGACGTCTGARACCATCGGGCGTAA
GCTCGCTTCCACCAACTCAGCCCTCAAGGCCGCCCAGGAGATGGGCAAGTTTGCAATTGAGGTYTTCAAG
CAGATCATGGCATGGATTTGGCCTTCTGAGGACCCGGTGCCTGCTCTGCTTTCCAACATGGAGCAGGCGG
TCATCAAGAATGAGTGCCAGCTTGAGAACCAGCTCACAGCCATGTTGCGGGATCGCAACGCTGGGGCCGA
GTTCCTGAAAGCACTTGATGAAGAAGAACAAGAGGTCCGCAGGATTGCGGCCAAGTGCGGGAACTCCGCC
ACCACGGGCACCACCAACGCCCTACTGGCTAGGATYAGCATGGCTCGTGCGGCCTTCGAGAAGGCCCGCG
CTGAGCAGACCTCCCGGGTTCGRCCCGTGGTGATCATGGTATCTGGCAGGCCCGGGATCGGGAAAACCTG
TTTCTGTCAAAACCTGGCAAAGAGGATTGCCGCCTCCCTTGGRGATGAGACCTCAGTCGGCATCATACCA
CGTGCTGACGTGGACCACTGGGATGCCTACAARGGCGCTAGGGTGGTCCTYTGGGATGATTTCGGCATGG
ACAACGTGGTGAAGGACGCTCTGCGGCTGCAGATGCTTGCTGACACATGCCCCGTCACGCTTAACTGTGA
CAGAATTGAGAACAAGGGKAAGATGTTTGATTCCCAGGTCATCATCATTACCACCAACCAGCAGACCCCA
GTGCCYCTGGATTATGTCAACCTGGAGGCGGTGTGCCGCCGCATAGATTTCCTGGTCTATGCTGAGAGTC
CTGTGGTGGATGCCGCTCGGGCCAGATCACCTGGCGATGTGGCTGCCGTTAARGCCGCCATGAGGCCAGA
TTACAGCCACATCAACTTCATTCTGGCCCCACAGGGTGGMTTTGACCGGCAGGGTAATACCCCCTATGGS
AAGGGCGTCACCAAGATCATCGGCGCCACCGCGCTCTGTGCAAGAGCGGTTGCTCTCGTCCATGAGCGCC
ATGATGACTTTGGCCTTCAGAACAAGGTCTATGATTTTGATGCTGGCAAGGTGACCGCCTTTAAGGCCAT
GGCGGCTGATGCCGGCATYCCYTGGTACAAGATGGCRGCRATYGGCTRYAAGGCCATGGGCTGCACCTGT
GTGGAGGAGGCCATGAATTTGCTGAAGGACTATGAGGTGGCCCCSTGCCAAGTGATCTACAAYGGGGCCA
CCTACAATGTCAGCTGYATCAARGGGGCCCCCATGGTWGAGAAGRTCAAGGAGCCYGAGYTGCCCAAGAC
AYTGGTCAACTGTGTCAGRAGRATCAAGGAGGCSCGCCTCCGYTGCTACTGCAGGATGGCCACAGATGTC
ATCACTTCYATCYTGCAGGCGGCTGGRACGGCYTTCTCTATYTACCATCARATTGAGAAGAAATCTAGGC
CTTCCTTTTATTGGGACCACGGTTACACCTACCGAGATGGCCCAGGTGCCTTTGACATCTTTGAGGATGA
CAACGATGGATGGTACCACTCTGAGRGCAAGAAGGGTAAGAATAAGAAAGGTCGGGGGCGGCCTGGTGTY
TTCAAGTCCCGTGGGCTCACGGATGAGGAGTACGATGAGTTCAAGAAGCGCCGCGAATCCAAGGGCGGCA
AGTACTCCATTGATGACTACCTCGCTGACCGCGAGCGAGAAGARGAGCTCCAGGAGCGAGATGAGGAGGA
GGCCATTTTCGGGGACGGCTTTGGCCTGAAAGCCACGCGCCGCTCCCGTAAGGCAGAGAGAGCCAGACTT
GGCCTGGTCTCGGGTGGTGACATCCGCGCCCGCAAGCCGATTGACTGGAATGTAGTTGGTCCCTCCTGGG
CCGACGATGATCGCCAGGTCGATTACGGTGAGAAGATCAACTTTGAGGCCCCAGTCTCCATCTGGTCCCG
TGTTGTCCAATTCGGCACGGGGTGGGGCTTCTGGGTCAGTGGCCATGTGTTCATCACHGCCAAGCACGTG
GCACCACCCAAGGGCACGGAGGTCTTTGGTCGTAAGCCCGAGGAATTCACTGTCACCTCCAGTGGGGATT
TCCTDAAATACCATTTCACCAGTGCCGTCAGGCCTGACATCCCTGCCATGGTTCTGGAGAACGGCTGCCA
GGAGGGCGTTGTTGCCTCAGTCCTCGTCAAGAGGGCTTCCGGCGAGATGCTCGCTCTGGCGGTCAGGATG
GGCTCACAGGCTGCCATCAAGATCGGCAACGCTGTGGTGCATGGGCAGACCGGCATGCTCTTAACTGGGT
CCAATGCCAAGGCCCAAGACCTCGGGACTATCCCGGGTGACTGTGGTTGCCCCTATGTTTACAAGAAGGG
AAACACCTGGGTTGTGATTGGGGTGCATGTGGCGGCTACTAGATCAGGCAACACCGTCATTGCCGCCACC
CATGGTGAGCCCACACTTGAGGCCCTAGAATTCCAGGGGCCCCCAATGCTCCCCCGCCCCTCTGGCACCT
ATGCTGGCCTCCCCATCGCCGACTATGGCGACGCCCCTCCCTTGAGCACCAAGACCATGTTCTGGCGCAC
CTCGCCAGAGAAGCTCCCCCCTGGAGCCTGGGAGCCAGCCTACCTTGGCTCCAAGGATGAGAGGGTGGAC
GGCCCTTCCTTACAGCAGGTCATGAGAGACCAACTCAAGCCCTACTCAGAGCCACGTGGCCTGCTCCCTC
CYCAGGAAATTCTGGACGCGGTTTGTGATGCCATCGAGAACCGCCTTGAGAACACCCTTGAGCCGCAGAA
GCCCTGGACATTCAAGAAGGCCTGYGAGAGYCTKGACAAGAAYACCAGCAGTGGRTACCCCTAYCACAAR
CAGAARAGCAAGGACTGGACGGGRACCGCCTTCATYGGCGAGCTCGGTGACCAGGCYACYCATGCCAACA
ACATGTATGAGATGGGTAAGTCCATGCGGCCCGTCTACACAGCTGCCCTCAAGGATGAGCTGGTCAAGCC
AGACAAGATCTACAAGAAGATAAAGAAGAGGTTGCTCTGGGGCTCTGACCTTGGCACCATGATTCGCGCC
GCCCGCGCTTTTGGCCCCTTCTGTGATGCCCTGAAAGAGACTTGTGTTCTTAATCCTGTYAGAGTGGGTA
TGTCGATGAACGAAGATGGCCCCTTCATCTTCGCGAGGCACGCCAAYTTCAGRTACCACATGGATGCAGA
TTACACCAGATGGGACTCCACCCAGCAGAGGGCYATCTTGAAGCGCGCCGGTGACATCATGGTGCGTCTC
TCCCCTGAGCCAGAGTTGGCTCGGGTGGTGATGGATGACCTCCTGGCCCCCTCGCTGCTGGACGTCGGCG
ACTATAAGATCGTCGTCGAAGAGGGGCTCCCGTCCGGGTGCCCCTGCACCACGCAGCTGAAYAGTCTGGC
CCATTGGATCCTGACCCTTTGTGCAATGGTTGAAGTGACCCGWGTTGACCCCGAYATYGTGATGCARGAR
TCTGAATTCTCCTTCTATGGTGATGACGAGGTGGTCTCGACCAACCTCGAATTGGATATGACCAAATACA
CCATGGCCCTGAAGCGGTACGGTCTTCTCCCGACCCGTGCGGACAAGGAGGAGGGCCCCCTGGAGCGTCG
CCAGACGCTGCAGGGCATCTCCTTCCTGCGCCGCGCAATAGTCGGTGACCAGTTTGGCTGGTATGGTCGC
CTCGACCGTGCTAGCATTGACCGCCAGCTTCTTTGGACWAAAGGACCCAATCACCARAACCCYTTTGAGA
CTCTCCCAGGACATGCTCAGAGACCCTCCCAATTGATGGCCCTGCTTGGTGAGGCTGCCATGCATGGTGA
AAAGTACTAYAGGACTGTGGCTTCCCGGGTCTCCAAGGAGGCCGCCCAGAGTGGGATAGAAATGGTGGTC
CCACGCCACCGGTCTGTTCTGCGCTGGGTGCGCTTTGGAACAATGGATGCTGAGACCCCGCAGGAACGCT
CAGCAGTCTTTGTGAATGAGGATGAGTGATGGCGCAGCGCCAAAAGCCAACGGCTCTGAAGCCAGCGGCC
AGGATCTTGTTCCTACCGCCGTTGAACAGGCCGTCCCCATTCAGCCCGTGGCTGGCGCGGCTCTTGCCGC
CCCCGCCGCCGGGCAAATCAACCAAATTGACCCCTGGATCTTCCAAAATTTTGTCCAATGCCCCCTTGGT
GAGTTTTCCATTTCACCTCGAAACACCCCAGGTGAAATACTGTTTGATTTGGCCCTCGGGCCAGGGCTCA
ACCCCTACCTCGCCCACCTCTCAGCCATGTACACCGGCTGGGTTGGGAACATGGAGGTTCAGCTGGTCCT
CGCCGGCAATGCCTTTACTGCTGGCAAGGTGGTTGTTGCCCTTGTACCACCCTATTTTCCCAAAGGGTCA
CTCACCACTGCTCAGATCACATGCTTCCCACATGTCATGTGTGATGTGCGCACCCTGGAGCCCATTCAAC
TSCCTCTTCTTGACGTGCGTCGAGTTCTTTGGCATGCTACCCAGGATCAGGAGGAATCTATGCGCCTGGT
CTGCATGCTGTACACGCCACTCCGCACAAACAGCCCGGGTGATGAGTCTTTTGTGGTCTCTGGCCGCCTT
CTTTCTAAGCCGGCGGCTGATTTCAATTTTGTATACCTGACCCCCCCCATTGAGAGAACCATCTACCGGA
TGGTCGACTTGCCCGTGTTGCAGCCGCGGCTGTGCACGCATGCTCGTTGGCCAGCCCCGATTTATGGCCT
CCTGGTGGACCCATCCCTCCCGTCCAAYCCCCAATGGCAGAATGGTAGAGTGCATGTTGATGGAACCCTC
CTCGGTACGACACCTGTCTCTGGGTCCTGGGTTTCCTGCTTTGCGGCTGAAGCTGCCTAYGAGTTTCAGT
CTGGCATTGGTGAGGTGGCAACTTTCACCCTGATTGAGCAGGATGGCTCTGCCTATGTCCCTGGTGACAG
GGCAGCACCCCTTGGCTACCCCGATTTCTCCGGGCAACTGGAGATTGAGGTGCAGACTGAGACCACCAAA
GCAGGTGACAAGCTGAAGGTGACCACCTTYGAGATGGTCCTTGGCCCCACCACCAACGTGGATCAAGCGC
CCTACCAGGGCAGGGTGTACGCYAGCCTAACGGCTGYGTCCTCCCTCGATCTGGTGGATGGCAGGGTTAG
GGCGGTTCCACGCTCTGTCTTTGGCTTCCAAGATGTGGTTCCTGAGTATAATGATGGCCTCCTTGTCCCC
CTTGCCCCCCCAATYGGCCCCTTYCTTCCTGGTGAGGTGCTTCTGAGGTTCCGGACCTACATGCGTCAGG
TTGACAGCTCTGACGCCGCTGCGGAAGCCATCGACTGCGCCCTTCCACAGGAATTCGTCTCGTGGTTTGC
GAGTAACGGATTCACGGTGCAGTCGGAGGCCCTGCTCCTTAGGTACAGGAACACCCTAACAGGGCAGCTG
CTGTTTGAGTGCAAGCTCTACAGCGAAGGCTACATCGCCCTGTCCTATCCGGGCTCAGGACCGCTCACCT
TCCCGACTGATGGCTTCTTCGAGGTTGTCAGTTGGGTCCCCCGCCTTTATCAATTGGCCTCTGTGGGAAG
CTTGGCAACAGGCCGAACACTCAAACAATAATGGCTGGTGCCCTCTTTGGAGCAATTGGAGGTGGCCTGA
TGGGTATAATTGGCAATTCCATCTCAAATGTTCAAAACCTTCAGGCAAATAAACAATTGGCTGCTCAGCA
ATTTGGTTAYAATTCTTCTTTGCTTGCAACGCAAATTCAGGCCCAGAAGGATCTCACTCTGATGGGGCAG
CAATTCAACCAGCAGCTCCAAGCCAACTCTTTCAAGCACGACTTGGAAATGCTCGGCGCCCAGGTGCAAG
CCCAGGCGCAGGCCCAGRAGAATGCCATCAACATCAAATCGGCACAACTCCAGGCCGCGGGCTTTTCAAA
GTCTGACGCCATTCGCCTGGCCTCGGGGCAGCAACCGACGAGGGCCGTCGACTGGTCGGGGACGCGGTAT
TACACCGCCAACCAGCCGGTCACGGGCTTCTCGGGTGGCTTYACCCCAAGTTACACTCCAGGTAGGCAAA
TGGCAGTCCGCCCTGTGGACACATCCCCTCTACCGGTCTCAGGTGGGCGCATGCCGTCCCTTCGTGGAGG
TTCCTGGTCTCCGCGTGACTACACGCCACAGACTCAAGGCACCTACACGAACGGTCGGTTCGYGTCCTTC
CCRAAGATCGGGAGTAGCAGGGCGTAGGTTGGAAGAGAAACCTTTCTGTGAAAATGATTTCTGCTTACTG
CTCTTTTCTTTTGGTAGTATTTAGATGCATTT
>Norwalk
(SEQ ID NO: 6)
GUGAAUGAUGAUGGCGUCGA
>MH218720.1 Norovirus GI isolate NORO_79_05_07_2014, complete genome
(SEQ ID NO: 7)
GTGAATGATGATGGCGTCGAAAGACGTCGTTGCAACTAATGTTGCAAGCAACAACAATGCTAACAACACT
AGTGCTACATCTCGGTTCTTATCGAGATTTAAGGGCTTAGGAGGCGGCGCAAGCCCCCCTAGCCCTATAA
AAATTAAAAGTACAGAAATGGCTCTGGGGTTAATTGGCAGAACGACCCCAGAATCAACGGGGACCGCTGG
CCCACCGCCCAAACAACAGAGAGACCGACCTCCTAGAACTCAGGAGGAGGTCCAGTACGGTATGGGGTGG
TCTGACAGGCCCATTGACCAGAACGTCAAATCATGGGAAGAGCTTGACACCACAGTTAAGGAAGAGATCC
TAGACAACCACAAAGAATGGTTTGACGCTGGTGGTTTGGGTCCTTGCACAATGCCTCCAACATATGAACG
GGTCAGGGATGACAGTCCGCCTGGTGAACAGGTTAAATGGTCCGCACGTGATGGAGTCAACATTGGAGTG
GAACGCCTCACAACAGTGAGTGGGCCTGAGTGGAATCTTTGCCCCTTACCCCCCATTGATTTGAGGAACA
TGGAACCAGCTAGTGAACCCACTATTGGAGATATGATAGAATTCTACGAAGGCCACATCTATCATTACTC
CATATACATTGGGCAAGGTAAGACAGTCGGCGTCCATTCTCCACAGGCGGCATTTTCAGTGGCTAGAGTG
ACCATCCAGCCCATAGCCGCTTGGTGGAGAGTTTGTTACATACCCCAACCCAAGCATAGACTGAGTTACG
ACCAACTCAAGGAACTAGAGAATGAGCCATGGCCATACGCGGCCATAACTAATAATTGTTTTGAATTCTG
CTGTCAAGTCATGAACCTTGAGGACACGTGGTTGCAAAGGCGACTGGTCACGTCGGGCAGATTCCACCAC
CCCACCCAGTCGTGGTCACAGCAGACCCCTGAGTTCCAACAAGATAGCAAGTTAGAGTTGGTTAGGGACG
CCATATTGGCTGCAGTGAATGGTCTTGTTTCGCAGCCCTTTAAGAACTTCTTGGGTAAACTCAAACCCCT
CAATGTGCTTAACATCCTGTCTAACTGTGATTGGACCTTCATGGGGGTGGTGGAAATGGTCATACTATTA
CTTGAACTCTTTGGTGTGTTCTGGAACCCGCCTGATGTATCCAATTTTATAGCGTCCCTTCTTCCTGATT
TCCATCTTCAGGGACCTGAAGACTTGGCACGAGATCTAGTCCCAGTGATTCTTGGTGGTATAGGATTGGC
CATTGGGTTCACCAGAGACAAAGTTACAAAGATCATGAAGAGTGCTGTGGATGGTCTTCGAGCTGCTACA
CAACTGGGACAGTATGGATTAGAAATATTCTCACTGCTCAAGAAGTACTTCTTTGGGGGGGACCAGACTG
AGCGCACCCTCAAAGGCATTGAGGCAGCAGTCATAGATATGGAGGTACTGTCCTCCACTTCAGTGACACA
GCTAGTGAGGGACAAACAGGCAGCAAAGGCCTATATGAACATCTTGGACAATGAAGAAGAGAAGGCCAGG
AAGCTCTCTGCTAAAAACGCTGACCCACATGTGATATCCTCAACAAATGCCCTAATATCGCGCATATCCA
TGGCACGATCTGCATTGGCCAAGGCCCAGGCTGAGATGACCAGTCGAATGCGACCAGTTGTCATTATGAT
GTGTGGTCCACCTGGGATTGGGAAGACCAAGGCTGCTGAGCACCTAGCTAAGCGTCTAGCCAATGAGATC
AGACCAGGTGGTAAGGTGGGGTTGGTTCCCCGTGAAGCTGTCGACCACTGGGACGGCTATCATGGTGAGG
AAGTGATGCTGTGGGATGACTATGGCATGACAAAAATACAAGACGACTGTAATAAACTCCAGGCCATTGC
TGATTCGGCCCCCCTCACATTAAATTGTGATAGGATTGAAAATAAAGGAATGCAGTTCGTTTCAGATGCA
ATAGTCATCACCACCAACGCCCCAGGCCCCGCCCCTGTGGACTTTGTCAACCTTGGACCAGTGTGTAGAC
GGGTCGACTTTTTGGTGTACTGCTCTGCCCCAGAGGTGGAGCAGATACGGAGAGTCAGCCCTGGCGACAC
ATCAGCACTGAAAGACTGCTTCAAGCCAGATTTCTCACATTTAAAAATGGAGCTGGCTCCACAAGGTGGG
TTCGATAATCAAGGGAACACACCGTTTGGCAGGGGCACCATGAAGCCAACAACCATTAATAGACTCCTCA
TACAAGCCGTGGCCCTTACCATGGAAAGGCAGGATGAGTTCCAGTTGCAGGGAAAGATGTATGACTTTGA
TGATGACAGGGTGTCAGCGTTCACCACCATGGCACGTGACAATGGCCTGGGCATCTTGAGCATGGCGGGT
CTAGGTAAGAAGCTACGCGGTGTCACAACGATGGAGGGCTTGAAGAATGCCCTGAAGGGATACAAAATTA
GTGCGTGCACAATAAAATGGCAGGCTAAAGTGTACTCACTAGAGTCAGATGGCAACAGTGTCAACATTAA
AGAGGAGAGGAACATCTTAACTCAACAACAACAGTCAGTGTGTGCTGCCTCTGTTGCGCTCACTCGCCTC
CGGGCTGCGCGTGCGGTGGCATACGCGTCATGCATCCAATCGGCTATAACCTCTATACTACAAATTGCTG
GCTCGGCCCTAGTGGTCAACAGAGCAGTGAAGAGAATGTTTGGCACGCGTACTGCCACCCTGTCCCTTGA
GGGCCCCCCCAGAGAACACAAGTGCAGGGTCCACATGGCCAAGGCCGCAGGAAAGGGGCCTATTGGCCAT
GATGATGTGGTAGAAAAGTATGGGCTTTGCGAAACTGAGGAGGACGAAGAAGTGGCCCACACTGAAATCC
CTTCTGCCACCATGGAGGGCAAGAATAAAGGGAAGAACAAGAAAGGACGTGGTCGGAAGAACAACTACAA
CGCCTTCTCCCGCAGGGGACTCAATGATGAAGAGTACGAAGAGTACAAGAAGATACGCGAGGAGAAAGGT
GGCAATTATAGCATACAGGAGTACCTAGAGGATAGGCAAAGGTATGAAGAAGAGCTAGCAGAGGTTCAAG
CAGGTGGAGATGGAGGAATCGGGGAAACTGAAATGGAAATCCGCCACAGAGTGTTCTACAAATCTAAGAG
TAGAAAGCATCACCAGGAAGAGCGACGCCAGCTAGGGCTGGTAACAGGTTCCGACATTCGGAAGAGAAAA
CCAATCGACTGGACCCCACCCAAGTCAGCATGGGCAGATGATGAGCGTGAGGTGGATTACAATGAGAAGA
TCAGTTTTGAGGCGCCCCCCACTTTATGGAGCAGAGTGACAAAGTTTGGGTCTGGATGGGGTTTCTGGGT
CAGCTCTACAGTCTTCATAACCACAACGCACGTCATACCAACCAGTGCGAAGGAATTCTTTGGTGAACCC
CTAACCAGCATAGCCATCCACAGGGCTGGTGAGTTCACTCTATTCAGGTTCTCAAAGAAAATTAGGCCTG
ACCTCACAGGTATGATCCTTGAGGAGGGTTGCCCCGAGGGCACAGTGTGTTCAGTACTAATAAAAAGGGA
CTCTGGTGAACTACTGCCATTGGCTGTAAGAATGGGCGCAATAGCATCAATGCGTATACAGGGCCGCCTT
GTCCATGGGCAGTCCGGCATGTTGCTCACCGGGGCCAATGCTAAGGGCATGGACCTTGGAACCATCCCAG
GAGACTGTGGGGCTCCTTATGTCTATAAGAGAGCCAACGACTGGGTGGTCTGTGGTGTACACGCTGCTGC
CACCAAATCAGGCAACACCGTTGTGTGCGCCGTTCAGGCCAGTGAAGGAGAAACCACGCTTGAAGGCGGT
GACAAAGGTCATTATGCTGGACATGAAATAATTAAGCATGGTTGTGGACCAGCCCTGTCAACCAAAACCA
AATTCTGGAAATCATCCCCCGAACCACTACCCCCTGGGGTCTATGAACCCGCCTACCTCGGGGGCCGGGA
CCCTAGGGTAACTGGCGGTCCCTCACTCCAACAGGTGTTGCGGGACCAGTTAAAGCCATTTGCTGAGCCA
CGAGGACGCATGCCAGAGCCAGGTCTCTTGGAGGCCGCAGTTGAGACTGTGACTTCATCATTAGAGCAGG
TTATGGACACTCCCGTTCCTTGGAGCTATAGTGATGCGTGCCAGTCCCTTGATAAGACCACTAGTTCTGG
TTTTCCCTACCACAGAAGGAAGAATGACGACTGGAATGGCACCACCTTTATCAGGGAGTTAGGGGAGCAG
GCAGCACACGCTAATAACATGTATGAACAGGCTAAAAGTATGAAACCCATGTACACGGCAGCACTTAAAG
ATGAACTAGTCAAACCAGAGAAGGTATACCAAAAAGTGAAGAAGCGCTTGTTATGGGGGGCAGACTTGGG
CACGGTGGTTCGGGCCGCGCGGGCTTTTGGTCCATTCTGTGATGCTATAAAATCCCACACAATCAAATTG
CCCATTAAAGTTGGAATGAATTCAATTGAGGATGGGCCACTGATCTATGCAGAACATTCAAAGTATAAGT
ACCATTTTGATGCAGATTACACAGCTTGGGATTCAACTCAAAATAGACAAATCATGACAGAGTCATTCTC
AATCATGTGTCGGCTAACTGCATCACCTGAACTAGCTTCAGTGGTGGCTCAAGATTTGCTTGCACCCTCA
GAGATGGATGTTGGCGACTATGTCATAAGAGTGAAGGAAGGCCTCCCATCTGGTTTTCCATGTACATCAC
AGGTTAATAGTATAAACCATTGGTTAATAACTCTGTGTGCCCTTTCTGAAGTAACTGGTCTGTCGCCAGA
TGTCATCCAGTCCATGTCATATTTCTCTTTCTATGGTGATGATGAAATAGTGTCAACTGACATAGAATTT
GATCCAGCAAAACTGACACAAGTCCTCAGAGAGTATGGACTTAAACCCACCCGCCCCGACAAAAGCGAGG
GCCCAATAATTGTGAGGAAGAGTGTGGATGGTTTAGTCTTTTTGCGTCGCACTATCTCCCGCGACGCCGC
AGGATTCCAGGGGCGACTGGACCGGGCATCCATTGAAAGGCAAATCTACTGGACTAGAGGACCCAACCAC
TCAGACCCTTTTGAGACCCTGGTGCCACATCAACAAAGGAAGGTCCAACTAATATCATTATTGGGTGAGG
CCTCACTGCATGGTGAAAAGTTTTACAGGAAGATTTCAAGTAAAGTCATCCAGGAGATTAAAACAGGGGG
CCTTGAAATGTATGTGCCAGGATGGCAAGCCATGTTCCGTTGGATGCGGTTCCATGACCTTGGTTTGTGG
ACAGGAGATCGCAATCTCCTGCCCGAATTTGTAAATGATGATGGCGTCTAAGGACGCCCCTCAAAGCGCT
GATGGCGCAAGCGGCGCAGGTCAACTGGTGCCGGAGGTTAATACAGCTGACCCCTTACCCATGGAACCTG
TGGCTGGGCCAACAACAGCCGTAGCCACTGCTGGGCAAGTTAATATGATTGATCCCTGGATTGTTAATAA
TTTTGTCCAGTCACCTCAAGGTGAGTTCACAATCTCTCCTAACAATACCCCCGGTGATATTTTGTTTGAT
TTACAATTAGGTCCACATCTAAACCCTTTCTTGTCACATTTGTCCCAAATGTATAATGGCTGGGTTGGGA
ACATGAGAGTCAGAATTCTCCTTGCTGGGAATGCATTCTCAGCTGGAAAGATTATAGTTTGTTGTGTCCC
CCCTGGCTTTACATCTTCTTCTCTCACCATAGCTCAGGCCACATTGTTTCCCCATGTAATTGCTGATGTG
AGAACCCTTGAGCCAATAGAAATGCCCCTCGAGGATGTACGCAATGTCCTCTATCACACCAATGATAATC
AACCAACAATGCGGTTGGTGTGTATGCTATACACGCCGCTCCGCACTGGTGGGGGGTCTGGTAATTCTGA
TTCCTTTGTAGTTGCTGGCAGGGTTCTCACAGCCCCTAGTAGCGACTTTAGTTTCTTGTTCCTTGTCCCG
CCTACCATAGAGCAGAAGACTCGGGCTTTCACTGTGCCTAATATCCCCTTGCAAACCTTGTCCAATTCTA
GGTTTCCTTCCCTCATCCAGGGGATGATTCTGTCCCCCGATGCATCTCAAGTGGTCCAATTCCAAAATGG
GCGCTGCCTTATAGATGGTCAACTCCTAGGCACTACACCCGCTACATCAGGACAGCTGTTCAGAGTAAGA
GGAAAGATAAATCAGGGAGCCCGCACACTTAACCTCACAGAGGTGGATGGTAAACCATTCATGGCATTTG
ATTCCCCTGCACCTGTGGGGTTCCCCGATTTTGGAAAATGTGATTGGCATATGAGAATCAGCAAAACCCC
AAACAACACAAGTTCAGGTGACCCCATGCGCAGTGTCAGCGTGCAAACCAATGTGCAGGGTTTTGTGCCA
CACCTGGGAAGTATACAATTTGATGAAGTGTTTAACCATCCCACAGGTGACTACATTGGCACCATTGAAT
GGATTTCCCAGCCATCTACACCCCCTGGAACAGATATTGATCTGTGGGAGATCCCCGATTATGGATCATC
CCTTTCCCAAGCAGCTAATCTGGCCCCCCCAGTATTCCCCCCTGGATTTGGTGAGGCCCTTGTGTACTTT
GTTTCTGCTTTCCCGGGCCCCAATAACCGCTCAGCCCCGAATGATGTACCCTGTCTTCTCCCTCAAGAGT
ACATAACCCACTTTGTCAGTGAACAAGCCCCAACGATGGGTGACGCAGCCTTACTGCATTATGTCGACCC
TGATACCAACAGGAACCTTGGGGAGTTCAAGCTATACCCTGGAGGTTACCTCACCTGTGTACCAAATGGG
GTAGGTGCCGGGCCTCAACAGCTTCCTCTTAATGGTGTTTTTCTCTTTGTTTCTTGGGTGTCTCGTTTTT
ATCAGCTTAAGCCTGTGGGAACAGCCAGTACGGCAAGAGGTAGGCTTGGAGTGCGCCGTATATAATGGCC
CAAGCCATCATAGGAGCAATTGCCGCGTCAGCTGCAGGCTCAGCATTGGGTGCGGGCATCCAGGCTGGTG
CCGAGGCTGCGCTTCAGAGTCAAAGATACCAACAAGACTTAGCCCTGCAAAGGAATACTTTTGAACATGA
CAAGGATATGCTTTCCTACCAGGTCCAGGCAAGTAATGCACTTTTGGCAAAGAATCTCAATACCCGCTAT
TCTATGCTTGTTGCAGGGGGTCTTTCTAGTGCTGATGCTTCTCGGGCTGTTGCTGGGGCCCCTGTAACAC
AATTGATTGATTGGAACGGCACTCGGGTTGCCGCCCCCAGATCAAGTGCAACAACTCTGAGGTCTGGTGG
TTTCATGGCAGTCCCCATGCCTGTTCAATCCAAATCTAAGGCCCTGCAATCCTCTGGGTTTTCTAATCCT
GCTTATGACACGTCCACAGTTTCTTCTAGGACTTCTTCTTGGGTGCAGTCACAGAATTCCCTGCGAAGTG
TGTCACCCTTTCATAGGCAGGCCCTTCAAACTGTATGGGTTACTCCACCTGGGTCTACTTCCTCTTCTTC
TGTTTCCTCAACACCTTATGGTGTTTTTAATACGGATAGGATGCCGCTATTCGCAAATTTGCGGCGTTAA
TGTTGTAATATAATGCAGCAGTGGGCACTATATTCAATTTGGTTTAATTAGTGAATAATTTGGCCATTGA
TTAGTGTTAA
>FCV
(SEQ ID NO: 8)
GUAAAAGAAAUUUGAGACAA
>KT970059.1 Feline calicivirus strain GX01-13, complete genome
(SEQ ID NO: 9)
ATGTCTCAAACTCTGAGCTTCGTGCTAAAAACCCACAGTGTCCGTAAGGACTTTGTGCACTCCGTCAAGT
TAACACTTGCTCGGAGGCGCGATCTTCAGTATCTTTATAACAAGCTTGCCCGCTCTATACGAGCGGAGGC
TTGTCCATCTTGTGCTAGTTACGACGTTTGTCCTAACTGCACCTCTAGTGACATTCCCGATGATGGTTCG
TCAACAAACTCGATTCCATCTTGGGATGACGTCACGAAAACTTCAACCTATTCCCTCTTACTCTCCGAGG
ATACATCTGATGAGCTTAGCCCTGATGATTTGGTTAACATTGCTTCCCACATCCGTAAGGCAATATCCTC
TCAGTCGCATCCTGCCAACAATGAGATGTGCAAAGAACAGCTCACCTCGTTGCTGACAGTGGCTGAGGCC
ATGTTGCCCCAACGATCGCGGTCAACAATCCCACTGCATCAGAAACACCAGGCAGCTCGATTGGAATGGA
GAGAAAAATTCTTTTCTAAACCTCTTGACTTCCTCCTTGAGAAACTTGGCATGTCTAAGGACATTCTACA
AACCACTGCTATTTGGAAGATTGTTTTGGAAAAGGCCTGCTACTGTAAATCTTATGGTGAACAATGGTTT
AATGCTGCAAAGGCAAAGCTCCGTGAGATCAAGGAATTCGAGGGAAGTACTTTAAAACCTTTAATTGGTG
CGTTTATTGACGGACTGCGGCTCATGACCGTCGATAATCCAAACCCTATTGGCTTCTTGCCAAAATTAAT
TGGCTTAGTTAAACCTCTAAATTTGGCAATGATAATTGACAACCATGAAAATACCATGTCAGGATGGGTT
GTAACCCTCACAGCAATCATGGAGCTGTACAACATTACTGAGTGTACAATTGATGTGATTACGGCGCTGA
TCACTGGATTCTATGACAAATTGGCAAAAGCTACCAAATTTTATAGTCAGGTTAAAGCTTTATTCACTGG
ATTTAGATCAGAGGAAGTGTCAAATTCATTTTGGTACATGGCAGCTGCAGTATTGTGCTACCTTATCACT
GGCTTGCTACCAAACAATGGCAGGCTTTCAAAAATCAAGGCCTGTTTGTCTGGTGCTTCGACGCTAGTAT
CTGGTATAATTGCCACACAAAAGCTTGCTGCAATGTTTGCCACTTGGAACTCCGAAACAATAGTTAATGA
ACTTTCAGCCAGGACTGTTGCGCTTTCGGAGCTTAACAACCCCACCACGACATCCGACACTGACTCAGTA
GAAAGACTACTAGAATTGGCTAAGATCTTACATGAAGAAATCAAAGTTCACACGTTGAATCCAATTATGC
AATCATACAACCCAATTCTCAGAAATTTGATGTCAACATTGGATGGTGTCATCACATCATGCAACAAACG
AAAAGCCATTGCTAAGAAGAGACCTGTTCCAGTATGTTATATACTAACTGGTCCACCAGGTTGTGGGAAA
ACAACAGCTGCTTTAGCATTGGCAAAGAAGTTGTCAGAACAAGAGCCATCTGTTATAAATTTGGATGTAG
ATCACCATGACACATACACTGGCAACGAAGTCTGCATCATTGATGAATTTGATTCGTCTGACAAGGTCGA
TTATGCAAATTTTGTTATTGGGATGGTTAATTCGGCACCCATGGTCTTAAATTGTGACATGCTTGAAAAC
AAGGGGAAGCTCTTTACCTCTAAATATATTATAATGACCTCTAATTCTGAAACTCCTGTTAAGCCCGGTT
CAAAGCGTGCCGGTGCATTCTATCGAAGGGTCACAATCATTGATGTCACAAACCCTTTGGTAGAGTCACA
CAAGCGCGCCAGACCTGGCACCTCTGTTCCTCGCAGTTGCTATAAGAAAAACTTCTCTCATCTGTCGCTT
GCTAAGCGTGGGGCTGAGTGTTGGAGCAAGGAGTATGTCCTTGACCCCAAGGGACTCCAGCATCAAAGCA
TTAAGGCCCCTCCGCCCACCTTCCTTAATATTGATTCTCTTGCTCAAACAATGATACAAGATTTCACACT
AAAGAACATGGCATTTGAGGCAGAGGAAGGATGCAGTGATCACCGGTATGGGTTTATCTGCCAGAAGGAG
GAAGTGGAAACAGTTCGCAGACTTCTTAATGCAATTAGGGTTAGGCTCAATGCAACTTTCACAGTCTGTG
TAGGGCCTGAAGCATCTAGTTCAGTGGGATGTACCGCTCACGTCTTAACACCAGATGAGCCGTTCAATGG
TAAAAGATTTGTGGTTTCTCGCTGTAATGAGGCGTCACTATCTGCATTAGAAGGCAACTGTGTCCAAACC
GCATTGGGTGTGTGCATGTCCAACAAGGATCTAACCCATTTGTGTCATTTCATAAGGGGGAAGATTGTCA
ATGATAGTGTCAGACTGGATGAACTACCCGCTAATCAACATGTGGTAACCGTTAACTCGGTGTTTGATTT
AGCCTGGGCTCTTCGCCGTCACCTGTCACTATCTGGACAGTTCCAAGCCATCAGAGCCGCATATGATGTG
CTTACTGTCCCCGATAAAATCCCTGCAATGTTAAGACACTGGATGGATGAGACTTCATTCTCTGATGAAC
ATGTCGTAACCCAATTCGTAACCCCTGGTGGTATAGTGATTCTTGAATCATGTGTTGGTGCTCGCATCTG
GGCCATTGGTCACAATGTGATCAGGGCTGGAGGTATCACCGCCACACCGACTGGGGGTTGCGTGAGATTA
ATGGGATTGTCGGCTCATACTATGCCATGGAGTGAAATCTTTAGGGAACTCTTCTCTCTTCTGGGGAAAA
TCTGGTCTAGTGTTAAAGTCTCCACTCTAGTTCTCACCGCTCTTGGAATGTACGCATCAAGATTCAGACC
AAAATCAGAGGCAAAAGGCAAGACAAAGAGCAAAATTGGCCCCTACAGAGGTCGTGGCGTTGCCCTTACC
GACGACGAGTATGATGAATGGAGGGAACACAATGCCACTAGAAAATTGGACTTATCTGTTGAAGATTTTC
TAATGCTAAGGCATCGCGCAGCACTTGGTGCTGATGATGCTGATGCTGTCAAATTCAGGTCTTGGTGGAG
CTCTAGATCAAGACTTGCTGATGATATAGAAGATGTCACCGTAATTGGCAAGGGTGGCGTTAAACATGAG
AAAATTAGAACAAACACTCTAAGAGCCGTTGATCGTGGCTACGATGTCAGCTTTGCTGAAGAATCTGGCC
CTGGAACCAAATTTCACAAGAATGCAATTGGCTCTGTCACTGATGCTTGTGGTGAACACAAGGGATACTG
TATCCATATGGGTCATGGTGTTTACGCTTCTGTTGCCCATGTGGTGAAAGGTGATTCATTCTTTCTTGGT
GAGAGGATCTTTGACTTGAAAACTAATGGTGAATTCTGTTGCTTTAGAAGCACAAGGGTACTCCCAAGTG
CAGCTCCTTTCTTTTCTGGAAAACCCACACGTGACCCATGGGGCTCTCCTGTTGCTACAGAGTGGAAGCC
AAAGCCCTACACAACAACATCTGGGAAAATTGTAGGGTGCTTCGCAACTACATCAACTGAAACCCACCCT
GGTGATTGTGGCCTGCCGTACATCGATGATTGTGGAAGAGTTACAGGGCTACATACAGGATCTGGAGGCC
CAAAGACCCCTAGTGCAAAATTAATTGTTCCATATGTCCACATTGATATGAAGGCCAAATCTGTCACTCC
CCAAAAGTATGATGTTACAAAACCTGACATCAGCTATAAAGGTTTAATTTGCAAACAATTGGACGAAATC
AGAATTATACCAAAGGGAACCCGGCTTCACGTATCTCCTGCTCACGTTGATGACTACGAAGAATGCTCTC
ACCAACCAGCATCCCTCGGTAGTGGTGATCCCCGATGTCCAAAATCTCTGACAGCTATTGTTGTTGATTC
CTTAAAACCTTACTGTGATAAAGTGGAAGGCCCTCCTCATGATATATTGCACAGAGTCCAGAAAATGCTG
ATTGATCACCTGTCTGGATTCGTCCCCATGAACATATCCTCTGAAACTTCTATGCTATCCGCATTTCACA
AATTGAATCATGACACATCTTGTGGACCTTACTTAGGTGGAAGGAAGAAAGATCATATGGTAAATGGTGA
ACCTGACAAAGCTCTCTTGGATCTCCTATCCTCAAAATGGAAATTGGCAACACAAGGGATTTCCCTCCCA
CACGAGTACACAATTGGTTTGAAAGACGAGCTGAGACCAGTGGAGAAAGTCGCTGAGGGAAAGAGGAGGA
TGATCTGGGGGTGTGATGTCGGTGTTGCTACTGTGTGTGCTGCTGCTTTCAAAGCTGTTAGTGATGCAAT
CACAGCAAATCATCAATATGGGCCTATTCAAGTTGGTATCAATATGGATAGTCCCAGTGTTGAGGCGCTG
TACCAACGGATCAAGAGCTTTGCCAAAGTCTTTGCAGTTGATTACTCCAAATGGGATTCGACTCAATCGC
CCCGTGTAAGTGCTGCCTCAATTGACATCCTGCGATACTTCTCTGACAGATCACCAATTGTTGATTCGGC
CACAAATACACTTAAAAGCCCACCAGTTGCTATTTTTAATGGAGTTGCTGTTAAGGTCACATCTGGTTTG
CCCTCCGAAATGCCCCTCACCTCTGTGATTAACTCTCTTAACCACTGTTTGTATGTTGGGTGTGCTATCG
TTCAATCTTTAGAGGCTAGGAATGTCCCTGTCACATGGAATTTGTTCTCCTCTTTTGACATGATGACTTA
TGGTGATGATGGTGTGTATATGTTTCCAATGATGTTTGCTAGTGTTAGTGACCAAATCTTTGGTAACCTT
TCTGCTTACGGCCTAAAACCAACCCGAGTTGACAAGACCGTTGGGGCTATTGAGCCAATTGACCCTGAGT
CAGTTGTCTTTCTAAAAAGAACAATCTCTAGAACTCCCCATGGTGTCCGAGGATTGTTGGATCGCAGTTC
AATAATTAGGCAGTTTTACTACATCAAAGGTGAAAACACAGATGATTGGAAAACCCCCCCAAAAACAATC
GATCCAACATCCCGTGGTCAGCAACTCTGGAATGCCTGCTTGTATGCTAGTCAACATGGAAGTGAGTTCT
ACAACAAGATTTACAAATTGGCTGTGAAGGCTGTTGAGTACGAAGGACTCCACCTTGACCCTCCTTCTTA
CAGTTCGGCTTTGGAACATTACAACAGCCAGTTCAATGGCGTGGAGGCGCGGTCCGATCAGATCAATATG
AGTGATGGTACCGCCCTACACTGTGATGTGTTCGAAGTTTGAGCATGTGCTCAACCTGCGCTAACGTGCT
AAAATACTATGATTGGGACCCCCACTTTAGATTGGTTATTAACCCCAACAAATTCTTACCCGTTGGTTTC
TGCAATAACCCTCTTATGTGTTGTTACCCTGAATTGCTTCCTGAATTTGGAACTGTGTGGGACTGTGATC
AATCCCCACTTCAAATCTACCTAGAGTCAATCCTTGGTGATGATGAGTGGTCTTCAACCTATGAAGCAAT
TGACCCTGTTGTGCCACCAATGCACTGGGACGAAGCTGGTAAGATCTTCCAGCCACACCCTGGTGTACTA
ATGCACCACATCATTGGTGAAGTCGCAAAGGCATGGGATCCGAATCTGCCTCTTTTCCGACTTGAGGCAG
ACGACAGTTCCGTAACAACGCCTGAACAGGGCACCGCTGTTGGTGGTGTGATTGCTGAGCCCAATGCACA
GATGGCAGCGGCCGCTGATACGGCTACTGGGAAAAGTGTCGACTCAGAATGGGAGAATTTCTTCTCATTC
CACACCAGTGTGAATTGGAGCACTTCTGAAACCCAAGGAAAGATTCTGTTTAAACAATCACTTGGTCCTC
TTCTAAACCCTTATCTGGAACATTTGTCTAAGCTATATGTTGCTTGGTCTGGGTCTATCGAAGTTAGATT
TTCTATCTCTGGTTCTGGTGTCTTTGGGGGGAAGCTCGCGGCTATTGTCGTACCGCCGGGGATTAATCCC
GTGGCGAGCACTTCAATGCTGCAATACCCGCATGTCCTATTTGATGCTCGTCAAGTAGAACCTGTCATTT
TTACTATTCCTGATCTTAGGAACTCGCTTTACCACTTAATGTCTGATACTGACACTACATCCTTGGTTAT
TATGATCTATAATGATTTGATTAACCCTTATGCTAATGATTCTAACTCCTCTGGATGCATTGTCACAGTA
GAGACTAAGCCTGGACCTGACTTCAAATTTCACCTCTTGAAACCACCTGGCTCAATGTTAACACATGGTT
CTGTACCGTCAGATTTGATTCCAAAATCATCCTCACTATGGATTGGCAACCGCTATTGGTCTGACATCAC
CGATTTCATTGTTCGTCCATTTGTGTTCCAGGCAAATCGTCACTTTGACTTTAATCAAGAGACAGCTGGT
TGGAGTACTCCAAGATTTCGGCCCATTAGTATTACCATCAGTCAAAAAGACGGTGCAAAACTTGGCACTG
GGATTGCCACTGATTTCATTGTACCTGGAATACCAGACGGATGGCCAGACACAACAATTGCAGAAGAACT
CATCCCCGCTGGTGACTATGCCATCACAAATTCAGCCAATAATGATATTGCCACAAAGGCTGCTTACGAG
GCAGCAGATGTTATCAAGAACAACACCAACTTTAGAGGTATGTACATTTGTGGCGCTCTTCAAAGAGCTT
GGGGAGACAAGAAAATTTCCAATACTGCTTTCATCACCACCGCTACAATCAGTAATAACTCCATCAAGCC
CTGTAACAAAATTGATCAAACAAAGATTACTGTGTTCCAAAACAACCATGTTGGTAGTGATGTACAAACA
TCTGATGACACACTAGCCTTGCTTGGTTATACGGGGATTGGAGAAGAAGCCATTGGGGCGAATAGGGAGA
AAGTTGTTCGCATCAGTGTTTTGCGTGAGGCTGGTGCACGCGGCGGGAATCACCCTATATTTTACAAAAA
CTCCATTAAATTAGGCTATGTAATTGGATCTATTGATGTGTTCAATTCTCAAATCTTGCACACGTCTAGG
CAATTGTCTCTTAACCATTATCTGTTGGCTCCTGACTCTTTTGCTGTTTATAGGATTATTGACTCTAATG
GTTCTTGGTTTGACATAGGTATTGATTCTGATGGATTCTCCTTTGTTGGTGTTTCTACCATTCCTCCGCT
AGAGTTTCCACTTTCTGCCTCCTTCATGGGAATACAATTGGCAAAGATTCGACTTGCCTCAAACATTAGG
AGTGCTATGACAAAATTATGAATTCAATATTAGGCCTTATTGACTCTGTAACTAACACAGTAAGTAAAGC
ACAACAAATTGAATTAGATAAAGCTGCACTTGGTCAAAATAGAGAACTTGCTTTAAAACGTATTAACTTG
GATCAGCAAGCTCTTAATAACCAGGTGTCGCAATTTAACAAACTTCTTGAGCAGAGGGTACAGGGCCCTA
TTCAGTCAGTTCGATTAGCTCGTGCTGCTGGATTCCGGGTTGACCCTTACTCATACACAAATCAAAATTT
TTATGATGACCAACTCAATGCAATTAGATTATCATATAGAAATTTGTTTAAAATGTAGAATGAATTTTAT
AATTTGGATTGATTGGATGTACCTCTTCGGGCTGTCGCTGCGCCTAACCCCAGGG
>PSaV
(SEQ ID NO: 10)
GUGAUCGUGAUGGCUAAUUG
>RHDV
(SEQ ID NO: 11)
GUGAAAAUUAUGGCGGCUAU
>Tulane
(SEQ ID NO: 12)
GUGACUAGAGCUAUGGAU
>BEC-NB
(SEQ ID NO: 13)
GUGAUUUAAUUAUAGAGAGA

### REFERENCES

1. WHO. Monogenetic Diseases. 2013; 1-7.
2. Gaudelli N M, Komor A C, Rees H A, Packer M S, et al. Programmable base editing of A·T to G·C in genomic DNA without DNA cleavage. Nature 2017; 551:464-471, DOI: 10.1038/nature24644.
3. Ran F A, Hsu P D P, Wright J, Agarwala V, et al. Genome engineering using the CRISPR-Cas9 system. Nat Protoc 2013; 8:2281-2308, DOI: 10.1038/nprot.2013.143.
4. Settings C. CRISPR in 2018: Coming to a Human Near You. MIT Technol Rev 2018; 1-7.
5. Komor A C, Kim Y B, Packer M S, Zuris J A, et al. Programmable editing of a target base in genomic DNA without double-stranded DNA cleavage. Nature 2016; 61:5985-91, DOI: 10.1038/nature17946.
6. Ran F A, Hsu P D, Lin C Y, Gootenberg J S, et al. Double nicking by RNA-guided CRISPR cas9 for enhanced genome editing specificity. Cell 2013; 154:1380-1389, DOI: 10.1016/j.cell.2013.08.021.
7. Tsai S Q, Wyvekens N, Khayter C, Foden J A, et al. Dimeric CRISPR RNA-guided FokI nucleases for highly specific genome editing. Nat Biotechnol 2014; 32:569-576, DOI: 10.1038/nbt.2908.
8. Keiji Nishida, Takayuki Arazoe, Nozomu Yachie, Satomi Banno, Mika Kakimoto, Mayura Tabata, Masao Mochizuki, Aya Miyabe, Michihiro Araki, Kiyotaka Y. Hara Z S and AK. Targeted nucleotide editing using hybrid prokaryotic and vertebrate adaptive immune systems. Science (80-) 2016; 8729: DOI: 10.1126/science.aaf8729.
9. Hu J H, Miller S M, Geurts M H, Tang W, et al. Evolved Cas9 variants with broad PAM compatibility and high DNA specificity. Nature 2018; 1-24, DOI: 10.1038/nature26155.
10. Kim Y B, Komor A C, Levy J M, Packer M S, et al. Increasing the genome-targeting scope and precision of base editing with engineered Cas9-cytidine deaminase fusions. Nat Biotechnol 2017; 3803: DOI: 10.1038/nbt.3803.
11. Gehrke J M, Cervantes O, Clement M K, Pinello L, et al. High-precision CRISPR-Cas9 base editors with minimized bystander and off-target mutations. 2018; DOI: 10.1101/273938.
12. Zafra M P, Schatoff E M, Katti A, Foronda M, et al. An optimized toolkit for precision base editing. bioRxiv 2018; 303131, DOI: 10.1101/303131.
13. Martin A S, Salamango D, Serebrenik A, Shaban N, et al. A fluorescent reporter for quantification and enrichment of DNA editing by APOBEC-Cas9 or cleavage by Cas9 in living cells. Nucleic Acids Res 2018; 1-10, DOI: 10.1093/nar/gky332.
14. Kim K, Ryu S-M, Kim S-T, Baek G, et al. Highly efficient RNA-guided base editing in mouse embryos. Nat Biotechnol 2017; 35:435-437, DOI: 10.1038/nbt.3816.
15. Aird E J, Lovendahl K N, Martin A St., Harris R S, et al. Increasing Cas9-mediated homology-directed repair efficiency through covalent tethering of DNA repair template. bioRxiv 2017; 231035, DOI: 10.1101/231035.
16. Zheng Y, Lorenzo C, Beal P A. DNA editing in DNA/RNA hybrids by adenosine deaminases that act on RNA. Nucleic Acids Res 2016; 45:3369-3377, DOI: 10.1093/nar/gkx050.
17. Punwani D, Kawahara M, Yu J, Sanford U, et al. Lentivirus Mediated Correction of Artemis-Deficient Severe Combined Immunodeficiency. Hum Gene Ther 2017; 28:112-124, DOI: 10.1089/hum.2016.064.
18. Logue E C, Bloch N, Dhuey E, Zhang R, et al. A DNA sequence recognition loop on APOBEC3A controls substrate specificity. PLoS One 2014; 9:1-10, DOI: 10.1371/journal.pone.0097062.
19. Komor A C, Zhao K T, Packer M S, Gaudelli N M, et al. Improved base excision repair inhibition and bacteriophage Mu Gam protein yields C:G-to-T:A base editors with higher efficiency and product purity. 2017; 1-10.
20. Gehrke J M, Cervantes O, Clement M K, Wu Y, et al. An APOBEC3A-Cas9 base editor with minimized bystander and off-target activities. Nat Biotechnol 2018; DOI: 10.1038/nbt.4199.
21. Shi K, Carpenter M A, Banerjee S, Shaban N M, et al. Structural basis for targeted DNA cytosine deamination and mutagenesis by APOBEC3A and APOBEC3B. Nat Struct Mol Biol 2016; 24: DOI: 10.1038/nsmb.3344.
22. Kosicki M, Tomberg K, Bradley A. Repair of double-strand breaks induced by CRISPR-Cas9 leads to large deletions and complex rearrangements. Nat Biotechnol 2018; DOI: 10.1038/nbt.4192.
23. Oka S, Leon J, Tsuchimoto D, Sakumi K, et al. MUTYH, an adenine DNA glycosylase, mediates p53 tumor suppression via PARP-dependent cell death. Oncogenesis 2014; 3:e121-10, DOI: 10.1038/oncsis.2014.35.
24. Michaels M L, Cruz C, Grollman A P, Miller J H. Evidence that MutY and MutM combine to prevent mutations by an oxidatively damaged form of guanine in DNA. Proc Natl Acad Sci USA 1992; 89:7022-7025, DOI: 10.1073/pnas.89.15.7022.
25. Luncsford P J, Manvilla B A, Patterson D N, Malik S S, et al. Coordination of MYH DNA glycosylase and APE1 endonuclease activities via physical interactions. DNA Repair (Amst) 2013; 12:1043-1052, DOI: 10.1016/j.dnarep.2013.09.007.
26. Yang H, Clendenin W M, Wong D, Demple B, et al. Enhanced activity of adenine-DNA glycosylase (Myh) by apurinic/apyrimidinic endonuclease (Ape 1) in mammalian base excision repair of an A/GO mismatch. Nucleic Acids Res 2001; 29:743-752.
27. Qi H, Zakian V A. The Saccharomyces telomere-binding protein Cdc13p interacts with both the catalytic subunit of DNA polymerase ?? and the telomerase-associated Est1 protein. Genes Dev 2000; 14:1777-1788, DOI: 10.1101/gad.14.14.1777.
28. Chen Y, Varani G. Engineering RNA-binding proteins for biology. FEBS J 2013; 280:3734-54, DOI: 10.1111/febs.12375.
29. Hess G T, Frésard L, Han K, Lee C H, et al. Directed evolution using dCas9-targeted somatic hypermutation in mammalian cells. Nat Methods 2016; 13:1036-1042, DOI: 10.1038/nmeth.4038.
30. Ryu S-M, Koo T, Kim K, Lim K, et al. Adenine base editing in mouse embryos and an adult mouse model of Duchenne muscular dystrophy. Nat Biotechnol 2018; 36:536-539, DOI: 10.1038/nbt.4148.
31. Kluesner M G, Nedveck D A, Lahr W S, Garbe J R, et al. EditR: A Method to Quantify Base Editing from Sanger Sequencing. 2018; 1:1-13, DOI: 10.1089/crispr.2018.0014.
32. Borja-Cacho D, Matthews J. NIH Public Access. Nano 2008; 6:2166-2171, DOI: 10.1021/n1061786n.Core-Shell.
33. Olspert et al., Protein-RNA linkage and posttranslational modifications of feline calicivirus and munne norovirus VPg proteins. PeerJ. 2016; 4: e2134. DOI: 10.7717/peerj.2134.
34. Anzalone, A. V., Randolph, P. B., Davis, J. R. et al. Search-and-replace genome editing without double-strand breaks or donor DNA. Nature (2019). DOI:10.1038/s41586-019-1711-4.

We claim:

1. A method for producing a genetically modified cell, the method comprising
(a) introducing into a cell one or more plasmids, mRNAs, or proteins encoding
(i) a universal precise base editor fusion protein comprising a deaminase fused to a Cas9 nuclease domain, wherein the Cas9 nuclease domain comprises a base excision repair inhibitor domain,
(ii) synthetic chimeric ssODN-ssORN duplex, wherein at least a portion of the ssORN is complementary to that of the Cas9 d-loop and comprises a nucleotide mismatch recognized by the base editor fusion protein; and
(ii) one or more gRNAs having complementarity to a target nucleic acid sequence to be genetically modified; and
(b) culturing the introduced cell under conditions that promote modification of the target nucleic acid sequence targeted by the one or more gRNAs, whereby the target nucleic acid sequence is modified by the base editor fusion protein and gRNAs relative to an unmodified cell, and whereby a genetically modified cell is produced.

2. The method of claim 1, wherein the base editor fusion protein is an upABE or an upBE.

3. The method of claim 1, wherein the base editor fusion protein comprises a dsRNA adenosine deaminase, the nucleotide mismatch is dA:C, and the Cas9 domain is fused to a PCV2 domain.

4. The method of claim 3, wherein the dsRNA adenosine deaminase comprises an amino acid substitution of an E to a Q at position 1008, as numbered relative to SEQ ID NO:1.

5. The method of claim 3, wherein the dsRNA adenosine deaminase comprises an amino acid substitution of an E to a Q at position 488, as numbered relative to SEQ ID NO:2.

6. The method of claim 3, wherein the dsRNA adenosine deaminase comprises the amino acid sequence set forth as SEQ ID NO:3.

7. The method of claim 3, wherein the base editor fusion protein is selected from hADAR1d^{E1008Q}-nCas9-PCV2 and hADAR2d^{E488Q}-nCas9-PCV2.

8. The method of claim 1, wherein the base editor fusion protein comprises a Apolipoprotein B mRNA-editing complex (APOBEC) cytidine deaminase and the nucleotide mismatch is dC:A.

9. The method of claim 1, wherein the cell is a T cell, Natural Killer (NK) cell, B cell, or CD34+ hematopoietic stem progenitor cell (HSPC).

10. The method of claim 1, wherein the one or more gRNAs is covalently linked to a murine norovirus 1 (MNV1) VPg protein.

11. The method of claim 1, wherein one of more gRNA comprises a 5′ extension comprising nucleic acid sequence complementary to a non R-loop strand.

12. The method of claim 1, wherein one of more gRNA comprises a 3′ extension comprising nucleic acid sequence complementary to a non R-loop strand.

13. A method for producing a genetically modified cell, the method comprising
(a) introducing into a cell one or more plasmids, mRNAs, or proteins encoding:
(i) a universal, precise staggered Cas9 editor comprising a nCas9 domain fused to MutY DNA glycosylase (MUTYH) and Apurinic Endonuclease 1 (APE1), wherein the nCas9 domain comprises a RuvC nuclease domain;
(ii) a synthetic chimeric ssODN-ssORN duplex, wherein at least a portion of the ssORN is complementary to that of the Cas9 d-loop and comprises a 8-Oxoguanine (OG); and
(ii) one or more gRNAs having complementarity to a target nucleic acid sequence to be genetically modified; and
(b) culturing the introduced cell under conditions that promote modification of the target nucleic acid sequence targeted by the one or more gRNAs, whereby the target nucleic acid sequence is modified by the staggered Cas9 editor relative to unmodified cell, and whereby a genetically modified cell is produced.

14. The method of claim 13, wherein the universal, precise staggered Cas9 editor comprises MUTYH-APE1-nCas9-PCV2.

15. The method of claim 13, wherein the cell is a T cell, Natural Killer (NK) cell, B cell, or CD34+ hematopoietic stem progenitor cell (HSPC).

16. A genetically modified cell obtained according to the method of claim 1.

17. A genetically modified cell obtained according to the method of claim 13.
